(19) United States
(12) Patent Application Publication
(10) Pub. No.: US 2004/0022820 A1
(43) Pub. Date: Feb. 5, 2004

(54) Reversed liquid crystalline phases with non-paraffin hydrophobes

(76) Inventor: David Anderson, Ashland, VA (US)

Correspondence Address: Whitham, Curtis & Christofferson, PC, Suite 340, 11491 Sunset Hills Road, Reston, VA, 20190, US

(21) Appl. No.: 10/460,659

(22) Filed: Jun. 13, 2003

Related U.S. Application Data
(63) Continuation-in-part of application No. 09/994,937, filed on Nov. 28, 2001.
(60) Provisional application No. 60/387,909, filed on Jun. 13, 2002.

(51) Int. Cl. A61K 35/78; A61K 7/00

(52) U.S. Cl. 424/401; 424/725

(57) ABSTRACT

Compounds which are otherwise difficult to solubilize, such as, for example, pharmaceutical actives difficult for the body to absorb, are solubilized into a composition using a solvent system that is a structured fluid. The structured fluid is a reversed cubic phase or reversed hexagonal phase material, or a combination thereof, which includes a polar solvent, a surfactant and a non-paraffinic liquid with a high octanol-water partition coefficient which does not qualify as a surfactant. The compositions thus formed are able to enhance absorption of drugs by the induction of local, transient nanopores in biomembrane absorption barriers and particularly those in which efflux mechanisms, such as those associated with P-glycoprotein and/or cytochrome 3A4, are active. The compositions and methods that are used for solubilizing pharmaceutical actives in structured fluids can simultaneously accomplish solubilization of difficultly soluble drugs and enhancement of absorption.

### BACKGROUND OF THE INVENTION

1. Field of the Invention

The present invention relates to the solubilization of compounds which are difficult to solubilize. In particular, the invention provides compositions, liquid crystalline solvent systems and methods for solubilizing such compounds. The invention also relates to the enhanced delivery of compounds through biomembrane absorption barriers, such as those found in cells, tissues, and organs.

2. Background of the Invention

A significant number of compounds with potential pharmaceutical activity and application are poorly soluble in water. Of these, many are also difficult to solubilize with simple liquids and even surfactant-rich phases that are approved for use as, and appropriate for use as, excipients in pharmaceutical products. Generally it is not always enough to solubilize the drug, even if it is in a non-toxic vehicle; the vehicle must lend itself to whatever transformation—e.g., encapsulation, enteric coating, freeze- or spray-drying—is required to arrive at the correct delivery format. For example, for pharmaceutical actives where the most desirable format is the pill form for oral delivery, still the most common drug format by far, most liquid solvents and even surfactants, unless encapsulated, will often be incompatible with the simplest tablet manufacturing procedures, since these procedures were generally developed with solids and powders in mind. Yet the application of these procedures to poorly-soluble drugs without the use of liquids or surfactants often yields a pill that achieves only a very limited bioavailability when administered. It should also be pointed out that while acidic (e.g., hydrochloride) or basic (e.g., sodium) salt forms of low-solubility drugs can often be soluble, such salts can precipitate in the body when they encounter pH conditions that deprotonate the acidic salt or protonate the basic salt.

For actives that are to be delivered by injection, solubilization of such compounds is made challenging by the very limited selection of solvents and structured liquids that are approved for injection at levels that would be required to solubilize the drug. Furthermore, water-miscible liquid excipients, most notably ethanol, are of limited value since, even when the drug is soluble in neat ethanol, it will often precipitate upon contact with water, either diluent water for injection or in the aqueous milieu of body fluids, such as blood.

Nanostructured liquid crystalline phases of the reversed type—namely reversed cubic and reversed hexagonal phases—can be of very low solubility in water, meaning that they maintain their integrity as vehicles upon entry into the body thus avoiding drug precipitation, and show a great deal of promise in fields such as controlled-release drug delivery. In work motivated by the amphiphilic nature and porous nanostructures or these materials, which could lead to very advantageous interactions with biomembranes—much more intimate than in the case of liposomes—and by the high viscosities of these phases which can be an important aid in processing, a number of techniques have been developed for encapsulating such phases. See, for example, U.S. Pat. No. 6,482,571 to Anderson which is herein incorporated by reference.

Previous attempts to use reversed cubic and reversed hexagonal phases in the solubilization of actives important in such fields as pharmaceutics have focused almost exclusively on three lipids having surfactant properties: monoglycerides, galactolipids, and phospholipids. However, monoglycerides are highly toxic in the bloodstream, and thus are not approved for use in such routes as injection, intraperitoneal, etc. Furthermore, monoglycerides hydrolyze during storage in the presence of water. And significantly, cubic phases based on monoglycerides have a very limited capacity for incorporating hydrophobes; for example, the addition of about 2% triglyceride to a monoolein-water cubic phase will destroy the cubic phase structure. Galactolipids are exceedingly expensive at present, requiring laborious extraction procedures and present to only low values in their biological sources. Furthermore, galactolipids are not presently approved for use in pharmaceutics (and in addition, the formation of a cubic phase generally requires a mixture of two galactolipids, making the regulatory hurdles even higher). The two most important phospholipids that have been investigated (and the only ones that are currently available at less than exhorbitant prices) are phosphatidylcholine (PC) and phosphatidylethanolamine (PE). Phosphatidylcholine suffers from two drawbacks in the present context: first, when combined with only water it does not form cubic phases at or near room temperature or body temperature, and second, its curvature properties limit its ability to promote the uptake of liquid crystalline particles containing the lipid, as discussed herein. Phosphatidylethanolamine, in contrast, does induce strong curvature in lipid bilayers containing the lipid, and thus can promote fusion between biomembranes and liquid crystalline particles containing the lipids (see below); however, PE is regarded as too toxic for general use in injectable or intraperitoneal products and is not even approved for use in orally-administered formulations. Thus, each of these surfactants suffer from fundamental limitations from the point of view of drug-delivery, particularly when the approach to using them is limited to binary (or pseudobinary) matrices, and thus there is clearly a need for a larger stable of liquid crystalline phases employing other surfactants and lipids.

Matrices based on lamellar phases, such as liposomes, can be of very low solubility, but generally rely on processes such as endocytosis or pinocytosis for interacting with cells, which are not only slow and inefficient but can result in an intact matrix trapped inside an endosome. Furthermore, the solubilization of difficultly-soluble pharmaceutical actives in liposomes has not met with great success.

In the literature studies of ternary surfactant systems, a majority of the surfactants investigated have been water-soluble, exhibiting normal rather than reversed phases and suffering from rapid dissolution in the body.

The solubilization of a poorly water soluble drug in a reversed cubic or reversed hexagonal liquid crystalline matrix is fundamentally a very promising approach from the point of view of drug-delivery, because absorption of the drug by lipid bilayers of the body, or passage across absorption barriers comprising lipid bilayers, can be facilitated by more intimate and favorable interactions between the bilayers of these matrices and bilayers of the body. However, another limitation in previous attempts to use reversed liquid crystalline phases in the solubilization of pharmaceutical actives has come about because of the tacit, and frequently incorrect, assumption that a drug of low solubility in water should be hydrophobic and should thus be soluble in lipid, or in a binary (or pseudo-binary) lipid-water system. In particular, most studies have been limited to matrices composed of only lipid (or surfactant) and water, or of lipid-water-paraffin systems, wherein the paraffinic third component has an apolar group which is one or more hydrocarbon chains. In such matrices, absent other bilayer components (components that partition preferentially into the bilayer), the hydrophobic portion of the bilayer usually is composed substantially of just liquid paraffin, namely the paraffinic chains of the lipid or surfactant, plus in some cases the paraffinic additive. This is not a robust milieu for the solubilization of complex pharmaceutical actives, which frequently have polar groups that are essential for the interaction of the drugs with their receptors. It is important to point out that this paraffinic milieu is not substantially changed by simply adding a paraffinic compound—and yet the literature has to a substantial degree taught away from the investigation of third components that are not paraffinic, making the tacit assumption that the hydrophobic group of the third component should closely match the hydrophobic group of the surfactant or lipid. Thus, the liquid crystals reported in pharmaceutically-acceptable ternary systems with insoluble surfactants (or lipids), water, and hydrophobic liquid additives have all used paraffinic additives such as fatty acids and glycerides of fatty acids. Furthermore, pharmaceutical acceptability aside, nearly every reported case has used a third component that is paraffinic, either a fatty acid derivative or an alkane or alkanol. These systems generally do not yield substantially higher drug solubilities than are reached with simple binary surfactant-water systems. Clearly, the paraffinic milieu of the bilayer interior is also substantially unchanged upon the addition of another surfactant, since surfactants by design have clean divisions between strongly-hydrophobic and strongly-hydrophilic portions of the molecule, such that the hydrophilic portion of the molecule is substantially excluded from the hydrophobic portion of the surfactant or lipid bilayer (or monolayer).

Reversed hexagonal phase compositions, and to an even larger extent reversed cubic phase compositions, are difficult enough to come by even without the constraint that they be pharmaceutically acceptable and useful, and especially difficult under that constraint. For a number of reasons, considerable insight is required to know how and where to look for these phases. Reversed hexagonal phases, and to an even greater extent reversed cubic phases, usually are found only in small regions of phase diagrams (with the exception of cubic phases based on certain monoglycerides; however, these have distinct disadvantages as described above), making them hard to locate. Finding them usually requires insight and the mixing and analysis of a large number of samples.

Presently the state of mathematical modeling of the thermodynamics of 2-component, and especially 3-component, surfactant systems is poorly developed, yielding a good deal of insight (mostly to the person who developed the model, and significantly less to those who simply read a publication of the model), but not permiting one to calculate the location of such phases a priori based on the molecular structures and properties of the components. (The situation is much better for one-component block copolymer systems; see for example Anderson, D M and Thomas, E L, Macromolecules 1988, Vol. 21, pp. 3221-3230. However, polymers are not well suited for solubilizing pharmaceutical actives.).

It would be highly desirable to have available reverse cubic and reverse hexagonal phase compositions, solvent systems, and methods for solubilizing compounds which are difficult to solubilize.

### SUMMARY OF THE INVENTION

It is an object of this invention to provide new pharmaceutically-acceptable compositions that exhibit superior capacity to solubilize difficultly-soluble actives.

It is a further object of this invention to provide new pharmaceutically-acceptable compositions for reversed cubic and reversed hexagonal phases that exist in equilibrium with water (or body fluids), such that portions or particles of these compositions maintain their integrity in the presence of aqueous solutions during production, in storage, and en route to their delivery site.

It is a further object of this invention to provide new pharmaceutically-acceptable compositions for reversed cubic and reversed hexagonal phases that are amenable to techniques that have been developed for producing highly functional microparticles from such phases.

It is a further object of this invention to provide new pharmaceutically-acceptable compositions for reversed cubic and reversed hexagonal phases that may exhibit an inherent tendency to promote absorption. The inventor has demonstrated the relationship between curvature properties of lipids and their tendency to promote porosity in bilayers, and their tendency to form reversed cubic and other reversed phases including L3 and reversed hexagonal phases. See Anderson D. M., Wennerstrom, H. and Olsson, U., J. Phys. Chem. 1989, 93:4532-4542. The tendency to induce or form porous microstructures is viewed in the present context as being advantageous with respect to drug-delivery, in that it promotes the integration of the administered lipidic microparticles with biomembranes that otherwise form barriers to absorption, in contrast with lamellar lipidic structures such as liposomes which show low curvature, and little or no porosity, and do not ordinarily show strong tendencies to integrate with biomembranes.

The present invention provides compositions comprising a structured fluid and a compound (the active, typically a pharmaceutical or nutriceutical active) present in the structured fluid, the compound being otherwise of sufficiently low solubility in water that more than about 100 ml of water are required to dissolve a therapeutic amount of the compound. The nanostructured fluid comprises a polar solvent, a surfactant, and a non-paraffinic liquid with a high octanol-water partition coefficient which does not qualify as a surfactant. The structured fluid comprises a reversed cubic phase or reversed hexagonal phase, or a combination thereof, composed of pharmaceutically acceptable components.

The invention further provides compositions each comprising a structured fluid, for the solubilization of compounds of low solubility in water, viz., wherein more than about 100 ml of water are required to dissolve a therapeutic amount of such compound. The nanostructured fluid comprises a polar solvent, a surfactant, and a non-paraffinic liquid with a high octanol-water partition coefficient which does not qualify as a surfactant. The structured fluid is a reversed cubic or reversed hexagonal liquid crystalline phase, or a combination thereof, composed of pharmaceutically acceptable components.

The invention further provides an internally administerable solvent system comprising a polar solvent, a surfactant, and a non-paraffinic liquid with a high octanol-water partition coefficient which does not qualify as a surfactant. The structured fluid is a reversed cubic or reversed hexagonal liquid crystalline phase, or a combination thereof, composed of pharmaceutically acceptable components.

The invention further provides an internally administerable solvent system comprising a polar solvent, a surfactant, and a non-paraffinic liquid with a high octanol-water partition coefficient which does not qualify as a surfactant, and a pharmaceutical active solubilized in this fluid. The structured fluid is a reversed cubic or reversed hexagonal liquid crystalline phase, or a combination thereof, composed of pharmaceutically acceptable components.

The present invention further provides a method for solubilizing a compound, the compound being otherwise of sufficiently low solubility in water that more than about 100 ml of water are required to dissolve a therapeutic amount of the compound in a nanostructured fluid. The nanostructured fluid comprises a polar solvent, a surfactant, and a non-paraffinic liquid with a high octanol-water partition coefficient which does not qualify as a surfactant. The structured fluid is a reversed cubic or reversed hexagonal liquid crystalline phase, or a combination thereof, composed of pharmaceutically acceptable components. The method comprises the steps of combining the compound with a solvent system and allowing the compound to be incorporated into said solvent system.

### DESCRIPTION OF THE PREFERRED EMBODIMENTS

The present invention provides compositions, solvent systems and methods which are useful for solubilizing compounds that are otherwise difficult to solubilize (i.e. they otherwise require more than about 100 ml of water to dissolve a therapeutic amount of the compound). The compositions, solvent systems and methods of the present invention are based on the surprising discovery that certain combinations of polar solvent, surfactant, and non-paraffinic liquid yield reversed cubic and reversed hexagonal phases that are pharmaceutically acceptable, capable of solubilizing difficultly-soluble compounds, and have porous microstructures that are capable of promoting absorption in the body.

The compositions of the embodiments given herein were found through a combination of insight and a great deal of laborious work making and characterizing samples. The insight that was applied came from a combination of two decades of experience in mapping phase behavior of three-component surfactant systems, and mathematical modeling that has been reported in a number of the current author's publications. See D M Anderson, S M Gruner and S Leibler, Proc. Nat. Acad. Sci. 1988, 85:5364-5368; D M Anderson, J C C Nitsche, H T Davis and E L Scriven, Adv. Chem. Phys., 1990, 77:337-396; P Ström and D M Anderson, Langmuir, 1992, 8:691-702; D M Anderson, H Wennerstrom and U. Olsson, J. Phys. Chem. 1989, 93:4532-4542; D M Anderson, Supplement to J. Physique, Proceedings of Workshop on Geometry and Interfaces, Aussois, France, September 1990, C7-1—C7-18; D. M. Anderson, P. Ström, in: Polymer Association Structures: Liquid Crystals and Microemulsions, 1988, pp. 204-224, ed. M. El-Nokaly, ACS Symposium Series; D M Anderson and Pelle Ström, Physica A, 1991, 176, 151-167; D M Anderson and E L Thomas, Macromolecules, 1988 21:3221-3230; H Wennerstrom and D M Anderson, in Statistical Thermodynamics and Differential Geometry of Microstructured Materials, IMA Volumes, Vol. 51, pp. 137-152, Springer-Verlag (1993); D M Anderson and H Wennerstrom, J. Phys. Chem. 1990, 94:8683-8694; D M Anderson, H T Davis, L E Scriven, J. Chem. Phys., 1989 91 (5):3246-3251; and E L Thomas, D M Anderson, C S Henkee and D Hoffman, Nature 1988, 334:598-601.

Definitions/Descriptions

In order to facilitate understanding of the present invention, the following definitions and descriptions of terms utilized herein are provided:

Dissolution: Is meant that a compound under consideration is dissolving or is undergoing dissolution.

Solubilize: Is meant to be essentially synonymous with the term “dissolve” or “dissolution”, though with a different connotation; a compound under consideration is solubilized in a liquid or liquid crystalline material if and only if the molecules of the compound are able to diffuse within the liquid or liquid crystalline material as individual molecules, and that such material with the compound in it make up a single thermodynamic phase. It should be borne in mind that slightly different connotations are associated with the terms “dissolve” and “solubilize”: typically the term “dissolve” is used to describe the simple act of putting a crystalline compound in a liquid or liquid crystalline material and allowing or encouraging that compound to break up and dissolve in the material, whereas the terms “solubilize” and “solubilization” generally refer to a concerted effort to find an appropriate liquid or liquid crystalline material that is capable of dissolving such compound.

Solubility of a surfactant; low solubility of a surfactant: There has been some confusion in the literature as to what is meant by the solubility of a surfactant, in particular when low-solubility surfactants (such as long-chain monoglycerides or phospholipids, to cite well-known examples) form liquid crystals at high concentrations. In the context of this invention, the solubility of a surfactant in water (at a given temperature and pressure) is determined by the phase behavior that occurs when adding the surfactant to water: the first molecules of surfactant will go into solution, as required by thermodynamics (i.e., no surfactant has a solubility that is rigorously zero; the solubility is always a finite, non-zero value), but if a limit is reached beyond which a liquid crystalline phase splits out, then the solubility limit has been reached, and the solubility of the surfactant is this limiting value. Thus, for example, the solubility of glycerol monooleate is usually—and correctly, in accordance with this definition—given as of order 10⁻¹³ M, despite the fact that it forms liquid crystalline phases in water at concentrations as high as 60%; indeed, a liquid crystalline phase forms with a composition of approximately 40% water and 60% monoolein as soon as the concentration of surfactant rises above the limiting concentration, or solubility, of 10⁻¹³ M. This low solubility fits intuitively with what is expected for a molecule such as monoolein, with its 18-carbon chain and relatively weak, uncharged polar head group. A surfactant is said to be of low solubility in water, in this disclosure, if the solubility limit according to this definition is less than about 1% by weight.

Matrix: In the present context, a “matrix” is meant to be a material that serves as the host material for an active compound or compounds.

Tunable: In the present context, the solubilizing properties of a matrix can be said to be “tunable” if the composition under consideration and/or structure of the matrix can be deliberately adjusted so as to substantially change the solubility of the active compound.

Difficultly-soluble: In the present context, a compound (e.g., a pharmaceutical or nutritional active) can be said to be difficultly-soluble in water if a single therapeutic dose of the active requires more than about 100 ml of water or buffer to solubilize it; it can be said to be difficultly-soluble in oil if a single therapeutic dose of the active cannot be solubilized in less than about 10 ml of octanol; or if the compound is otherwise less than 5% by weight soluble in soybean oil. The choice of octanol as one standard is based on its broad usage in connection with the octanol-water partition coefficient. The choice of soybean oil is based on the broad usage of liquid triglycerides such as soybean oil, sesame oil, and peanut oil, in pharmaceutics and the fact that these liquid triglycerides all behave very similarly with respect to solubilization of actives.

Pharmaceutical active: a compound or agent that exhibits biological activity, including nutritional, nutriceutical and/or pharmacological activity.

Excipients: compound and mixtures of compounds that are used in pharmaceutical formulations that are not the active drugs themselves.

Pharmaceutically-acceptable: a composition in which each excipient is approved by the Food and Drug Administration or is otherwise safe for use in a pharmaceutical formulation intended for internal use; this also includes compounds that are major components of approved excipients, which are known to be of low toxicity taken internally. A listing of approved excipients, each with the various routes of administration for which they are approved, was published by the Division of Drug Information Resources of the FDA in January, 1996 and entitled “Inactive Ingredient Guide”. The existence of a Drug Master File at the FDA is additional evidence that a given excipient is acceptable for pharmaceutical use. In the present context, this listing includes, as approved for internal use (oral, injectable, intraperitoneal, etc.), such excipients as: benzyl benzoate, peppermint oil, orange oil, spearmint oil, ginger fluid extract (also known as essential oil of ginger), thymol, vanillin, anethole, cinnamon oil, cinnamaldehyde, clove oil, coriander oil, benzaldehyde, poloxamer 331 (Pluronic 101), polyoxyl 40 hydrogenated castor oil—indeed, a wide range of surfactants with polyethyleneglycol head groups—calcium chloride and docusate sodium. Absent from the list are a number of apolar or very weakly polar liquids that are more associated with applications as fuels or organic solvents: liquid hydrophobes including toluene, benzene, xylene, octane, decane, dodecane, and the like. In contrast, the hydrophobes and polar hydrophobes that are approved as excipients tend to be natural extracts which have a history of use in foods, nutriceuticals, or pharmaceutics—or early precursors to these disciplines. Examples of compounds that are major components of approved excipients and known to be of low toxicity include: linalool, which is a major component of coriander oil and is the subject of extensive toxicity studies demonstrating its low toxicity; vanillin, which is a major component of the approved excipient ‘flavor vanilla’ and is one of the major taste components of vanilla-flavored foods and pharmaceutical formulations; and d-limonene, which is a major component of the approved excipient ‘essence lemon’ approved for use in oral formulations and has extensive everyday applications in which its low toxicity is important. By “component” we mean a molecule that is present as a distinct and individual molecule in a mixture, not as a chemical group in a larger molecule; for example, methanol (methyl alcohol) would not be considered to be a component of methyl stearate. It should be noted that within a given series of compounds of varying molecular weight, there is very frequently a considerable difference between the approval status of the liquids in the series and the solids (at room temperature or body temperature); it happens commonly that the solids are approved for internal use whereas the liquids are not. One reason for this is that liquids inherently have a greater potential for disrupting biological membranes than do solids, which tend to behave more as inerts. However, for the purposes of this invention, it is liquids which have a greater value by far as the hydrophobe, for the obvious reason that liquids are far better solvents than solids (though this is not to say that solids are useless, since for example menthol (m.p. about 42° C.) is soluble in many surfactant-water mixtures and can aid in the dissolution of many actives. For the purposes of this invention, a compound will be considered to be a pharmaceutically-acceptable excipient if it can be created by a simple ion-exchange between two compounds that are on the FDA listing; thus, for example, calcium docusate is to be considered a pharmaceutically-acceptable excipient since it is a natural result of combining sodium docusate and calcium chloride (in the presence of water, for example).

Paraffinic, non-paraffinic: a compound will be considered paraffinic in the context of this invention if and only if it contains an acyclic, uninterrupted saturated hydrocarbon chain segment at least 6 carbons in length, not counting any carbon atoms that are branched from this main chain. While the number 6 is to some extent arbitrary, it matches the criterion (cited below) given by Laughlin for the minimum chain length for self-association to occur; the shortest surfactant chains are 6 carbons in length discounting branches, as for example in sodium hexane sulfonic acid and in sodium 2-ethylhexyl sulfosuccinate (sodium docusate). A compound is then considered non-paraffinic if it is free of such chain segments with length 6 or greater. We note that the presence of long, unsaturated hydrocarbon chains on a compound can still qualify the compound as paraffinic under this definition, if the unsaturation nonetheless leaves segments of saturated chain length greater than 6; for example, oleic acid would qualify as paraffinic because, although it contains a double bond at position 9, there is an uninterrupted segment of 8 carbons in a fully saturated configuration.

Amphiphile: an amphiphile can be defined as a compound that contains both a hydrophilic and a lipophilic group. See D. H. Everett, Pure and Applied Chemistry, vol. 31, no. 6, p. 611, 1972. It is important to note that not every amphiphile is a surfactant. For example, butanol is an amphiphile, since the butyl group is lipophilic and the hydroxyl group hydrophilic, but it is not a surfactant since it does not satisfy the definition, given below. There exist a great many amphiphilic molecules possessing functional groups which are highly polar and hydrated to a measurable degree, yet which fail to display surfactant behavior. See R. Laughlin, Advances in liquid crystals, vol. 3, p. 41, 1978.

Surfactant: A surfactant is an amphiphile that possesses two additional properties. First, it significantly modifies the interfacial physics of the aqueous phase (at not only the air-water but also the oil-water and solid-water interfaces) at unusually low concentrations compared to non-surfactants. Second, surfactant molecules associate reversibly with each other (and with numerous other molecules) to a highly exaggerated degree to form thermodynamically stable, macroscopically one-phase, solutions of aggregates or micelles. Micelles are typically composed of many surfactant molecules (10's to 1000's) and possess colloidal dimensions. See R. Laughlin, Advances in liquid crystals, vol. 3, p. 41, 1978. Lipids, and polar lipids in particular, often are considered as surfactants for the purposes of discussion herein, although the term ‘lipid’ is normally used to indicate that they belong to a subclass of surfactants which have slightly different characteristics than compounds which are normally called surfactants in everyday discussion. Two characteristics which frequently, though not always, are possessed by lipids are, first, they are often of biological origin, and second, they tend to be more soluble in oils and fats than in water. Indeed, many compounds referred to as lipids have extremely low solubilities in water, and thus the presence of a hydrophobic solvent may be necessary in order for the interfacial tension-reducing properties and reversible self-association to be most clearly evidenced, for lipids which are indeed surfactants. Thus, for example, such a compound will strongly reduce the interfacial tension between oil and water at low concentrations, even though extremely low solubility in water might make observation of surface tension reduction in the aqueous system difficult; similarly, the addition of a hydrophobic solvent to a lipid-water system might make the determination of self-association into nanostructured liquid phases and nanostructured liquid crystalline phases a much simpler matter, whereas difficulties associated with high temperatures might make this difficult in the lipid-water system.

Indeed, it has been in the study of nanostructured liquid crystalline structures that the commonality between what had previously been considered intrinsically different—‘lipids’ and ‘surfactants’—came to the forefront, and the two schools of study (lipids, coming from the biological side, and surfactants, coming from the more industrial side) came together as the same nanostructure observed in lipids as for surfactants. In addition, it also came to the forefront that certain synthetic surfactants such as dihexadecyldimethylammonium bromide which were entirely of synthetic, non-biological origin, showed ‘lipid-like’ behavior in that hydrophobic solvents were needed for convenient demonstration of their surfactancy. On the other end, certain lipids such as lysolipids, which are clearly of biological origin, display phase behavior more or less typical of water-soluble surfactants. Eventually, it became clear that for the purposes of discussing and comparing self-association and interfacial tension-reducing properties, a more meaningful distinction was between single-tailed and double-tailed compounds, where single-tailed generally implies water-soluble and double-tailed generally oil-soluble.

Thus, in the present context, any amphiphile which at very low concentrations lowers interfacial tensions between water and hydrophobe, whether the hydrophobe be air or oil, and which exhibits reversible self-association into nanostructured micellar, inverted micellar, or bicontinuous morphologies in water or oil or both, is a surfactant. The class of lipids simply includes a subclass of surfactants which are of biological origin.

Lipid: in the context of this invention, a lipid is considered to be a molecule formed by a hydrophilic moiety and a lipophilic moiety, the two linked together by bonds sufficiently flexible to yield a rather independent behavior. See Luzzati, in Biological Membranes, Chapter 3, page 72 (D. Chapman, ed. 1968). The terms “lipid” and “surfactant” are utilized interchangeably herein.

Hydrophobe: in the context of this invention, a compound is considered to be a hydrophobe if and only if it is a compound of high octanol-water partition coefficient—preferably about 10 greater or and more preferably about 100 or greater—and does not satisfy the definition of a surfactant given herein. According to this definition, a compound can be a hydrophobe and still contain one or more polar groups, provided that the polar groups are not sufficiently dominant to yield true surfactant behavior. However, if a compound has a polar group that is operative as a surfactant head group according to Laughlin (see below), then this is not considered a hydrophobe in the present context. For example, sodium cholate is not a hydrophobe because it contains a carboxylate ion, operative as a head group; indeed, sodium cholate is known to form surfactant microstructures such as micelles.

It should be noted that a compound will not be a surfactant unless it contains at least one of the groups listed herein that qualify as surfactant head groups, according to the publication of Laughlin cited. This is discussed in detail in the section entitled “Chemical criteria”.

Chemical criteria: In the case of surfactants, a number of criteria have been tabulated and discussed in detail by Robert Laughlin for determining whether a given polar group is functional as a surfactant head group, where the definition of surfactant includes the formation, in water, of nanostructured phases even at rather low concentrations. R. Laughlin, Advances in Liquid Crystals, 3:41, 1978.

The following listing given by Laughlin gives some polar groups which are not operative as surfactant head groups—and thus, for example, an alkane chain linked to one of these polar groups would not be expected to form nanostructured liquid or liquid crystalline phases—are: aldehyde, ketone, carboxylic ester, carboxylic acid (in the free acid form), isocyanate, amide, acyl cyanoguanidine, acyl guanylurea, acyl biuret, N,N-dimethylamide, nitrosoalkane, nitroalkane, nitrate ester, nitrite ester, nitrone, nitrosamine, pyridine N-oxide, nitrile, isonitrile, amine borane, amine baloborane, sulfone, phosphine sulfide, arsine sulfide, sulfonamide, sulfonamide methylimine, alcohol (monofunctional), ester (monofunctional), secondary amine, tertiary amine, mercaptan, thioether, primary phosphine, secondary phosphine, and tertiary phosphine.

Some polar groups which are operative as surfactant head groups, and thus, for example, an alkane chain linked to one of these polar groups would be expected to form nanostructured liquid and liquid crystalline phases, are:

a. Anionics: carboxylate (soap), sulfate, sulfamate, sulfonate, thiosulfate, sulfinate, phosphate, phosphonate, phosphinate, nitroamide, tris(alkylsulfonyl)methide, xanthate;

b. Cationics: ammonium, pyridinium, phosphonium, sulfonium, sulfoxonium;

c. Zwitterionics: ammonio acetate, phosphoniopropane sulfonate, pyridinioethyl sulfate;

d. Semipolars: amine oxide, phosphoryl, phosphine oxide, arsine oxide, sulfoxide, sulfoximine, sulfone diimine, ammonio amidate.

Laughlin also demonstrates that as a general rule, if the enthalpy of formation of a 1:1 association complex of a given polar group with phenol (a hydrogen bonding donor) is less than 5 kcal, then the polar group will not be operative as a surfactant head group.

In addition to the polar head group, a surfactant requires an apolar group, and again there are guidelines for an effective apolar group. For alkane chains, which are of course the most common, if n is the number of carbons, then n must be at least 6 for surfactant association behavior to occur, although at least 8 or 10 is the usual case. Interestingly octylamine, with n=8 and the amine head group which is just polar enough to be effective as a head group, exhibits a lamellar phase with water at ambient temperature, as well as a nanostructured L2 phase. Wamheim, T., Bergenstahl, B., Henriksson, U., Malmvik, A. -C. and Nilsson, P. (1987) J. of Colloid and Interface Sci. 118:233. Branched hydrocarbons yield basically the same requirement on the low n end; for example, sodium 2-ethylhexylsulfate exhibits a full range of liquid crystalline phases. Winsor, P. A. (1968) Chem. Rev. 68:1. However, the two cases of linear and branched hydrocarbons are vastly different on the high n side. With linear, saturated alkane chains, the tendency to crystallize is such that for n greater than about 18, the Krafft temperature becomes high and the temperature range of nanostructured liquid and liquid crystalline phases increases to high temperatures, near or exceeding 100° C.; in the context of the present invention, for most applications this renders these surfactants considerably less useful than those with n between 8 and 18. With the introduction of unsaturation or branching in the chains, the range of n can increase dramatically. The case of unsaturation can be illustrated with the case of lipids derived from fish oils, where chains with 22 carbons can have extremely low melting points, due to the presence of as many as 6 double bonds, as in docosahexadienoic acid and its derivatives, which include monoglycerides, soaps, etc. Furthermore, polybutadiene of very high MW is an elastomeric polymer at ambient temperature, and block copolymers with polybutadiene blocks are well known to yield nanostructured liquid crystals. Similarly, with the introduction of branching, one can produce hydrocarbon polymers such as polypropyleneoxide (PPO), which serves as the hydrophobic block in a number of amphiphilic block copolymer surfactants of great importance, such as the Pluronic series of surfactants. Substitution of fluorine for hydrogen, in particular the use of perfluorinated chains, in surfactants generally lowers the requirement on the minimal value of n, as exemplified by lithium perfluourooctanoate (n=8), which displays a full range of liquid crystalline phases, including an intermediate phase which is fairly rare in surfactant systems. As discussed elsewhere, other hydrophobic groups, such as the fused-ring structure in the cholate soaps (bile salts), also serve as effective apolar groups, although such cases must generally be treated on a case-by-case basis, in terms of determining whether a particular hydrophobic group will yield surfactant behavior.

Polar-apolar interface: In a surfactant molecule, one can find a dividing point (or in some cases, 2 points, if there are polar groups at each end, or even more than two, as in Lipid A, which has seven acyl chains and thus seven dividing points per molecule) in the molecule that divide the polar part of the molecule from the apolar part. In any nanostructured liquid phase or nanostructured liquid crystalline phase, the surfactant forms monolayer or bilayer films; in such a film, the locus of the dividing points of the molecules describes a surface that divides polar domains from apolar domains; this is called the “polar-apolar interface,” or “polar-apolar dividing surface.” For example, in the case of a spherical micelle, this surface would be approximated by a sphere lying inside the outer surface of the micelle, with the polar groups of the surfactant molecules outside the surface and apolar chains inside it. Care should be taken not to confuse this microscopic interface with macroscopic interfaces, separating two bulk phases, that are seen by the naked eye.

Structured fluid: Particularly useful mixtures from the point of view of microencapsulation and drug-delivery that occur in systems containing surfactant and polar solvents are structured fluids. For the purposes of this disclosure, a structured fluid is taken to be a fluid that has structural features on a length scale much larger than atomic dimensions, in particular fluids such as nanostructured liquids, nanostructured liquid crystals, and emulsions. Examples include L1, L2 and L3 phases, lyotropic liquid crystalline phases, emulsions, and microemulsions.

Lyotropic liquid crystalline phases. Lyotropic liquid crystalline phases include the normal hexagonal, normal bicontinuous cubic, normal discrete cubic, lamellar, reversed hexagonal, reversed bicontinuous cubic, and reversed discrete cubic liquid crystalline phases, together with the less well-established normal and reversed intermediate liquid crystalline phases.

The nanostructured liquid crystalline phases are characterized by domain structures, composed of domains of at least a first type and a second type (and in some cases three or even more types of domains) having the following properties:

a) the chemical moieties in the first type domains are incompatible with those in the second type domains (and in general, each pair of different domain types are mutually incompatible) such that they do not mix under the given conditions but rather remain as separate domains; (for example, the first type domains could be composed substantially of polar moieties such as water and lipid head groups, while the second type domains could be composed substantially of apolar moieties such as hydrocarbon chains; or, first type domains could be polystyrene-rich, while second type domains are polyisoprene-rich, and third type domains are polyvinylpyrrolidone-rich);

b) the atomic ordering within each domain is liquid-like rather than solid-like, lacking lattice-ordering of the atoms; (this would be evidenced by an absence of sharp Bragg peak reflections in wide-angle x-ray diffraction);

c) the smallest dimension (e.g., thickness in the case of layers, diameter in the case of cylinders or spheres) of substantially all domains is in the range of nanometers (viz., from about 1 to about 100 nm); and

d) the organization of the domains conforms to a lattice, which may be one-, two-, or three-dimensional, and which has a lattice parameter (or unit cell size) in the nanometer range (viz., from about 5 to about 200 nm); the organization of domains thus conforms to one of the 230 space groups tabulated, for example, in the International Tables of Crystallography, and would be evidenced in a well-designed small-angle x-ray scattering (SAXS) measurement by the presence of sharp Bragg reflections with d-spacings of the lowest order reflections being in the range of 3-200 nm.

Reversed hexagonal phase: In surfactant-water systems, the identification of the reversed hexagonal phase differs from the above identification of the normal hexagonal phase in only two respects:

1. The viscosity of the reversed hexagonal phase is generally quite high, higher than a typical normal hexagonal phase, and approaching that of a reversed cubic phase. And,

2. In terms of phase behavior, the reversed hexagonal phase generally occurs at high surfactant concentrations in double-tailed surfactant/water systems, often extending to, or close to, 100% surfactant. Usually the reversed hexagonal phase region is adjacent to the lamellar phase region which occurs at lower surfactant concentration, although bicontinuous reversed cubic phases often occur in between. The reversed hexagonal phase does appear, somewhat surprisingly, in a number of binary systems with single-tailed surfactants, such as those of many monoglycerides (include glycerol monooleate), and a number of nonionic PEG-based surfactants with low HLB.

As stated above in the discussion of normal hexagonal phases, the distinction between ‘normal’ and ‘reversed’ hexagonal phases makes sense only in surfactant systems, and generally not in single-component block copolymer hexagonal phases.

Reversed cubic phase: The reversed bicontinuous cubic phase is characterized by:

In surfactant-water systems, the identification of the reversed bicontinuous cubic phase differs from the above identification of the normal bicontinuous cubic phase in only one respect. In terms of phase behavior, the reversed bicontinuous cubic phase is found between the lamellar phase and the reversed hexagonal phase, whereas the normal is found between the lamellar and normal hexagonal phases; one must therefore make reference to the discussion above for distinguishing normal hexagonal from reversed hexagonal. A good rule is that if the cubic phase lies to higher water concentrations than the lamellar phase, then it is normal, whereas if it lies to higher surfactant concentrations than the lamellar then it is reversed. The reversed cubic phase generally occurs at high surfactant concentrations in double-tailed surfactant/water systems, although this is often complicated by the fact that the reversed cubic phase may only be found in the presence of added hydrophobe (‘oil’) or amphiphile. The reversed bicontinuous cubic phase does appear in a number of binary systems with single-tailed surfactants, such as those of many monoglycerides (include glycerol monooleate), and a number of nonionic PEG-based surfactants with low HLB.

It should also be noted that in reversed bicontinuous cubic phases, though not in normal, the space group #212 has been observed. This phase is derived from that of space group #230. As stated above in the discussion of normal bicontinuous cubic phases, the distinction between ‘normal’ and ‘reversed’ bicontinuous cubic phases makes sense only in surfactant systems, and generally not in single-component block copolymer bicontinuous cubic phases.

Hydrophobes of Utility in the Present Invention.

It follows from the definitions given above that a non-paraffinic hydrophobe must in fact be a hydrophobic compound (Kow>10, preferably >100) which is not a surfactant, i.e., in which any polar group on the molecule is on a par with the following groups listed by Laughlin as being not operative as a surfactant head group: aldehyde, ketone, carboxylic ester, carboxylic acid (in the free acid form), isocyanate, amide, acyl cyanoguanidine, acyl guanylurea, acyl biuret, N,N-dimethylamide, nitrosoalkane, nitroalkane, nitrate ester, nitrite ester, nitrone, nitrosamine, pyridine N-oxide, nitrile, isonitrile, amine borane, amine haloborane, sulfone, phosphine sulfide, arsine sulfide, sulfonamide, sulfonamide methylimine, alcohol (monofunctional), ester (monofunctional), secondary amine, tertiary amine, mercaptan, thioether, primary phosphine, secondary phosphine, and tertiary phosphine. Of these groups, preferred groups for the polar group(s) are, given in approximate order from most preferred to less preferred: alcohol (monofunctional, including phenolic), carboxylic acid, aldehyde, amide, secondary amine, and tertiary amine. The distinction as a preferred group is based mainly on issues of low toxicity, low reactivity, sufficient polarity, and on the lack of tendency to yield high-melting point compounds.

For the pharmaceutically-acceptable hydrophobe of the current invention, there are a number of low-toxicity hydrophobic liquids with polar groups, many of which have a history of safe use in pharmaceutical and/or food products, that could be used. These include essential oils of plant origin, as well as a number of other liquids that are listed on FDA's list entitled Inactive Ingredients for Currently Marketed Drug Products and/or the appropriate sections of the Food Additives Status List. Among these are: benzyl benzoate, cassia oil, castor oil, cyclomethicone, polypropylene glycol (of low MW), polysiloxane (of low MW), cognac oil (ethyl oenanthate), lemon balm, balsam of Peru, cardamom oleoresin, estragole, geraniol, geraniol acetate, menthyl acetate, eugenol, isoeugenol, petigrain oil, pine oil, rue oil, trifuran, annato extract, turmeric oleoresin, and paprika oleoresin.

Essential oils from plant sources (including their extracts and components, and mixtures thereof) comprise a rather large and chemically diverse group of liquids that include many low-toxicity hydrophobes with polar groups. The term “essential oils” is intended to include essential oils from the following sources:

allspice berry, amber essence, anise seed, arnica, balsam of Peru, basil, bay, bay leaf, bergamot, bois de rose (rosewood), cajeput, calendula (marigold pot), white camphor, caraway seed, cardamon, carrot seed, cedarwood, celery, german or hungarian chamomile, roman or english chamomile, cinnamon, citronella, clary sage, clovebud, coriander, cumin, cypress, eucalyptus, fennel, siberian fir needle, frankincense (olibanum oil), garlic, rose geranium, ginger, grapefruit, hyssop, jasmine, jojoba, juniper berry, lavender, lemon, lemongrass, lime, marjoram, mugwort, mullein flower, myrrh gum, bigarade neroli, nutmeg, bitter orange, sweet orange, oregano palmarosa, patchouly, pennyroyal, black pepper, peppermint, petitegrain, pine needle, poke root, rose absolute, rosehip seed, rosemary, sage, dalmation sage, santalwood oil, sassafras (saffrole-free), spearmint, spikenard, spruce (hemlock), tangerine, tea tree, thuja (cedar leaf), thyme, vanilla extract, vetivert, wintergreen, witch hazel (hamamelia) extract, or ylang ylang (cananga).

The following are components of essential oils:

2,6-dimethyl-2,4,6-octatriene; 4-propenylanisole; benzyl-3-phenylpropenoic acid; 1,7,7-trimethylbicyclo[2.2.1]heptan-2-ol; 2,2-dimethyl-3-methylenebicyclo[2 .2 .1 ]heptane; 1,7,7-trimethylbicyclo[2.2.1]heptane; trans-8-methyl-n-vanillyl-6-nonenamide; 2,2,5-trimethylbicyclo[4.1.0]hept-5-ene; 5-isopropyl-2-methylphenol; p-mentha-6,8-dien-2-ol; p-mentha-6,8-dien-2-one; beta-caryophyllene; 3-phenylpropenaldehyde; 3,7-dimethyl-6-octenal; 3,7-dimethyl-6-octen-1-ol; 4-allylanisole; ethyl 3-phenylpropenoic acid; 3-ethoxy-4-hydroxybenzaldehyde; 1,8-cineole; 4-allyl-2-methoxyphenol; 3,7,11-trimethyl-2,6,10-dodecatrien-1-ol; 1,3,3-trimethylbicyclo[2.2.1]heptan-2-ol; 1,3,3-trimethylbicyclo[2.2.1]heptan-2-one; trans-3,7-dimethyl-2,6-octadien-1-ol; trans-3,7-dimethyl-2,6-octadien-1-yl acetate; 3-methyl-2-(2-pentenyl)-2-cyclopenten-1-one; p-mentha-1,8-diene; 3,7-dimethyl-1,6-octadien-3-ol; 3,7-dimethyl-1,6-octadien-3-yl acetate; p-menthan-3-ol; p-menthan-3-one; methyl 2-aminobenzoate; methyl-3-oxo-2-(2-pentenyl)-cyclopentane acetate; methyl 2-hydroxybenzoate; 7-methyl-3-methylene-1,6-octadiene; cis-3,7-dimethyl-2,6-octadien-1-ol; 2,6,6-trimethylbicyclo[3.1.19 hept-2-ene; 6,6-dimethyl-2-methylenebicyclo[3 1.1]heptane; p-menth-4(8)-en-3-one; p-menth-1-en-4-ol; p-mentha-1,3-diene; p-menth-l-en-8-ol; and 2-isopropyl-5-methylphenol.

Especially preferred non-surfactant hydrophobes, due to a favorable combination of good drug-solubilizing properties, low toxicity, low water solubility, useful temperature range as a liquid, history of use, and compatibilty with (or induction of) cubic phases, are: benzyl benzoate, estragole, eugenol, isoeugenol, linalool, and the following essential oils: balsam of Peru, basil, bay, bois de rose (rosewood), carrot seed, clovebud, eucalyptus, ginger, grapefruit, hyssop, lemon, mugwort, myrrh gum, bitter orange, oregano, palmarosa, patchouly, peppermint, petitgrain, rosemary, santalwood oil, spearmint, thuja (cedar leaf), thyme, vanilla, and ylang ylang (cananga).

Polar solvents. The polar solvents employed in the practice of the present invention include but are not limited to:

a. water;

b. glycerol;

c. ethylene glycol or propylene glycol;

d. ethylammonium nitrate;

e. one of the acetamide series: acetamide, N-methyl acetamide, or dimethylacetamide;

f. low-molecular weight polyethylene glycol (PEG);

g. a mixture of two or more of the above.

Preferred polar solvents are water, glycerol, ethylene glycol, N-methylacetamide, dimethylacetamide, and polyethylene glycol, since these are considered of low toxicity. However, with the compositions given herein that rely on PEGylated (ethoxylated) surfactants (such as Arlatone and Pluronics), glycerol is generally not compatible.

Advantages and Unique Properties.

The cubic and hexagonal phases described herein have a number of unique properties, and significant advantages over cubic phases that have been described in the literature, particularly as relate to their potential application in drug-delivery, cosmeceutics, and nutriceuticals.

To begin with, the problems and limitations associated with the lipids used in the prior art for making reversed cubic and reversed hexagonal phases for solubilizing actives that were discussed above, including toxicity and regulatory problems, limited ability to incorporate hydrophobes that are useful for solubilizing actives (in the case of monoglycerides), expense (in the case of galactolipids), and inappropriate phase behavior, are substantially eliminated in the compositions reported in this disclosure. The classes of ethoxylated castor oil derivatives, Pluronics, ethoxylated tocopherols, docusates, and sorbitan fatty acid monoesters used in the embodiments of this invention all have members that are approved for injectable formulations. Thus, focusing on the latter class for a moment, it is notable that no monoglyceride (glycerol fatty acid monoester) is approved for injection, whereas the sorbitan fatty acid monoester sorbitan monopalmitate appears on the 1996 FDA “Inactive Ingredient Guide” as being approved for use in injectable products. This is a striking difference between these two classes of compounds.

With the incorporation of a non-paraffinic hydrophobe, particularly one containing at least one polar group, the ability of these cubic phases to solubilize difficultly-soluble drugs and actives is greatly improved. As discussed elsewhere herein, most pharmaceutical compounds that are water-insoluble nevertheless contain at least one, usually several, and frequently four or more polar groups. Since most lipid-water cubic phases reported in the literature, as well as those reported here, are based on lipids that do not have polar groups in the acyl chains (with the exception of the castor oil derivatives), and thus have very low concentrations of polar groups in the interior of the lipid bilayer where water-insoluble compounds are presumably solubilized, most simple lipid-water systems are poorly suited for solubilizing water-insoluble compounds with a number of polar groups. The incorporation of a non-paraffinic hydrophobe, preferably containing at least one polar group into the liquid crystal, and thus into the lipid bilayer, dramatically changes the concentration of polar groups in the bilayer, increasing its effective polarity, making for more favorable enthalpic interactions with drug molecules. Compounds of these sorts are particularly preferred if the hydrophobe is of low molecular weight, about 500 or less, and especially if the MW is about 250 or less, so that it takes on more of a true “solvent-like” nature, with entropic effects more strongly favoring dissolution of the hydrophobe in the bilayer, and the drug in the hydrophobe-lipid environment.

It is important to point out that while certain fatty acids and derivatives thereof can be used in the formation of reversed liquid crystalline phases, they are clearly less effective than non-paraffinic hydrophobes in the modulation of the bilayer interior milieu. Infinitely more effective are non-paraffinic hydrophobes, in particular those that are more compact, such as aromatic compounds in particular (e.g., zingerone, a major component of ginger oil), or compounds such as carvone (a major component of oil of spearmint), which has a combination of low MW (150.2), unsaturation, branching, and polar groups. In contrast, the simple fatty acids, particularly medium- and long-chain fatty acids and their close relatives will tend to simply add more paraffin to the hydrophobic portion of the bilayer, and not cause a fundamental change in the local milieu as would accompany the addition of, for example, cinnamaldehyde.

Distinct advantages possessed by individual surfactants or classes of surfactants are reported in the Examples below.

It is also important to point out that there is much to be gained simply by virtue of enlarging the repertoire of cubic phase and hexagonal phase compositions. In a given application of liquid crystals in phairmaceutics or another field, typically there are many criteria that must be simultaneously satisfied, and this calls for a stable of compositions each with its own particular strengths. For example, for any given pharmaceutical active, there are usually a handful of hydrophobes that outperform all the other available hydrophobes in terms of solubilizing that active to a high loading, and the available surfactants and lipids vary in their ability to tolerate the solubilizing effect of these hydrophobes (which often liquify what are otherwise liquid crystalline phases), and yield ternary liquid crystalline phases capable of solubilizing the active to a substantial loading. This will vary from drug to drug, and call for a different liquid crystal composition as this varies. Beyond this are issues of enhancing absorption, toxicity, and compatibility with other features and processes in the overall formulation such as encapsulation with a particular coating, pH and ionic conditions, etc.

Compounds that are of Low Solubility in Both Water and Lipid.

It is a mistake to tacitly assume that a compound that is water-insoluble should be soluble in lipid—in other words, that the terms “hydrophobic” and “lipophilic” are equivalent. It is true that when a water-insoluble molecule can be fairly cleanly divided into a very small number (generally 3 or less) of well-defined polar and apolar regions, then the compound is often soluble in lipid. However, particularly in the world of pharmaceutical actives, it is common to find a larger number of polar and apolar groups dispersed in a single molecule. In such cases, one strategy for solubilizing the drug in a lipid bilayer system is to introduce non-paraffinic hydrophobes and particularly those that present polar groups in the bilayer interior.

For example, consider the structure of dantrolene. As one moves along the length of the molecular structure diagram of dantrolene, one finds: a polar group (nitro group), low-polarity group (aromatic ring), moderately-polar group (furanyl ring), polar group (methylamino), and finally a hydantoin group which is charged or uncharged depending on pH. This compound has a solubility of approximately 150 mg/L in water, and even its sodium salt has a solubility on the order of 300 mg/L. Further, its solubility in simple phospholipid-water systems is also very low, too low to be of practical pharmaceutical importance. It is difficult to imagine a configuration of the drug in a lipid bilayer that would avoid direct contact between at least one of the polar groups with an acyl chain of the phospholipid.

The case of paclitaxel is even more demonstrative of molecules that cannot be neatly divided into polar and apolar sections. The molecule has 47 carbon atoms, includes 3 distinct aromatic rings, and has an exceedingly low solubility in water. However, a significant number of polar groups are present: one amide group, 3 hydroxyls, 4 ester bonds, another carbonyl group, and an cyclopropoxy ring. Table 1 lists representative pharmaceutical compounds from some of the major therapeutic categories which are of low solubility in water, and tabulates the number of polar groups on the molecule. The table demonstrates that many, if not most, water-insoluble drugs contain at least 3 polar groups, and would be expected to have low solubility in a simple lipid-water mixture. The incorporation of a non-paraffinic hydrophobe in accordance with the present invention remedies this. Examination of the chemical structure of each of these compounds furthermore reveals that the polar groups are spread throughout the molecule, so that only in rare cases would the molecule be able to situate itself in a simple (lipid-water) bilayer with an orientation analogous to that of a surfactant. Most of these drugs listed are also problematic when attempts are made to solubilize the drug in water by converting the drug to a salt, such as a hydrochloride, or sodium salt for example; for example, some would precipitate at the pH of the body milieu, others would decompose, etc.

TABLE 1

| Therapeutic Category | Compound | A | B | C | D | E | F | G | H | Total |
|---|---|---|---|---|---|---|---|---|---|---|
| ACE inhibitor | Enalapril | 1 | | 1 | 1 | 1 | | | | 4 |
| beta-Adrenergic | Albuterol | 1 | 2 | | | | 1 | | | 4 |
| agonist | | | | | | | | | | |
| beta-Adrenergic | Sulfinalol | 1 | 1 | | | | 1 | | 2 | 5 |
| blocker | | | | | | | | | | |
| Anabolic | Nandrolone | | 1 | | | 1 | | | | 2 |
| Analgesic (narcotic) | Morphine | 1 | 1 | | | | 1 | | 1 | 4 |
| Analgesic (non- | Aspirin | | | 1 | | 1 | | | | 2 |
| narcotic) | | | | | | | | | | |
| Androgen | Testosterone | | 1 | | | 1 | | | | 2 |
| Anesthetic | Hexobarbitol | | | | 2 | 1 | | | | 3 |
| (intravenous) | | | | | | | | | | |
| Anorexic | Cyclexedrine | 1 | | | | | | | | 1 |
| Anthelmintic | Niclosamide | | | | 1 | | 1 | | 1 | 3 |
| (cestodes) | | | | | | | | | | |
| Anthelmintic | Mebendazole | 2 | | | 1 | 1 | | | | 4 |
| (nematodes) | | | | | | | | | | |
| Anthelmintic | Amphotalide | 1 | | | 1 | 1 | | | 1 | 4 |
| (schistosoma) | | | | | | | | | | |
| Antiacne | Retinoic acid | | | 1 | | | | | | 1 |
| Antiamebic | Emetine | 1 | | | | | | | 4 | 5 |
| Antianginal | Nifedipine | 1 | | | | 2 | | | 1 | 4 |
| Antiarrhythmic | Quinidine | 2 | 1 | | | | | | 1 | 4 |
| Antibiotic | Chloramphenicol | | 1 | | 1 | | | | 1 | 3 |
| (amphenicol) | | | | | | | | | | |
| Antibiotic (ansamycin) | Rifamide | | 2 | | 2 | 3 | 2 | | 4 | 13 |
| Antibiotic (lactam) | Ampicillin | 1 | | 1 | 2 | | | | 1 | 5 |
| Antibiotic (macrolide) | Erythromycin A | 1 | 5 | | | 2 | | | 4 | 12 |
| Antibiotic | Tetracycline | 1 | 4 | | 1 | 2 | 1 | | | 9 |
| (tetracycline) | | | | | | | | | | |
| Antibacterial | Ciprofloxacin | 3 | | 1 | | 1 | | | | 5 |
| (quinolone) | | | | | | | | | | |
| Antibacterial | Sulfamoxole | 2 | | | | | | | 2 | 4 |
| (sulfonamide) | | | | | | | | | | |
| Antibacterial (sulfone) | Dapsone | 2 | | | | | | | 1 | 3 |
| Anticholinergic | Atropine | 1 | 1 | | | 1 | | | | 3 |
| Anticoagulant | Warfarin | | 1 | | | 2 | | | | 3 |
| Anticonvulsant | Nitrazapem | 1 | | | 1 | | | | 1 | 3 |
| Antidepressant | Zometapine | 4 | | | | | | | | 4 |
| Antidiabetic | Glyburide | | | | 3 | | | | 2 | 5 |
| Antidiarrheal | Uzarin | | 7 | | | 1 | | | 1 | 9 |
| Anti-inflammatory | Aspirin | | | 1 | | 1 | | | | 2 |
| Antineoplastic | Taxol | | 3 | | 1 | 5 | | | 1 | 10 |
| Antineoplastic | Etiposide | | 2 | | | 1 | 1 | | 8 | 12 |
| Skeletal muscle | Dantrolene | 1 | | | 2 | | | | 2 | 5 |
| relaxant | | | | | | | | | | |

A = amino;
B = hydroxyl;
C = carboxyl;
D = amide;
E = carbonyl;
F = phenolic;
G = cation
H = other

Table 2 also lists candidate pharmaceutical agents for use in the present invention.

TABLE 2

| Pharm Class | Generic Name | Trade Name |
|---|---|---|
| Anabolic steroid | Nandrolone decanoate | Androlone |
| Analgesic | Fentanyl citrate | Sublimaze |
| Androgen | Testosterone | Testoderm, etc |
| Anthelmintic | Albendazole | Albenza |
| Antibiotic, antineoplastic | Doxorubicin | Rubex |
| Antibiotic, antineoplastic | Epirubicin | Ellence |
| Antibiotic, antineoplastic | Idarubicin | Idamycin |
| Antibiotic, antineoplastic | Valrubicin | Valstar |
| Anticholinergic | Oxybutinin | Ditropan |
| Antifungal | Amphotericin B | Fungizone, etc. |
| Antihypertensive | Enalaprilat | Vasotec |
| Antimitotic | Docetaxel | Taxotere |
| Antimitotic | Paclitaxel | Taxol |
| Antimitotic | Vinblastine | Velban |
| Antimitotic | Vincristine | Oncovin |
| Antimitotic | Vinorelbine | Navelbine |
| Antineoplastic | Batimastat | |
| Antiplatelet | Eptifibatide | Integrilin |
| Antiplatelet | Tirofiban | Aggrastat |
| Antipsychotic, anesthetic | Droperidol | Droperidol, Inapsine |
| Antiviral | Acyclovir | Zovirex; Valtrex |
| Antiviral | Pentafuside | none |
| Antiviral | Saquinavir | Fortovase |
| Asthma anti-inflammatory | Cromolyn | Intal |
| CNS stimulant | Doxapram | Dopram |
| DNA topoisomerase inhibitor | SN-38 (Irinotecan) | Camptosar |
| DNA topoisomerase inhibitor | Topotecan | Hycamtin |
| Enzyme inhibitor | Hemin | Panhematin |
| Epipodophyllotoxin | Daunorubicin | Daunorubicin; DaunoXome* |
| Epipodophyllotoxin | Teniposide | Vumon |
| Folate antagonist | Trimetrexate | Neutrexin |
| Gastric antisecretory | Octreotride | Sandostatin |
| Hormone | Leuprolide | Lupron, Viadur |
| Immunosuppressant | Clyclosporin A | Sandimmune |
| Inotropic agent | Milrinone lactate | Primacor |
| Narcotic agonist/antagonist | Buprenorphine | Buprenex |
| Narcotic agonist/antagonist | Nalbuphine | Nubain |
| Platinum complex | Carboplatin | Paraplatin |
| Platinum complex | Cisplatin | PlatinolAQ |
| Platinum complex | Mitoxantrone | Novantrone |
| Sex hormone | Estradiol | Kestrone, etc. |
| Sex hormone | Hydroxyprogesterone | Hylutin |
| Thyroid hormone | L-Thyroxine | Levothroid, etc. |
| TNF inhibitor (arthritis) | Etanercept | Enbrel |
| Urinary cholinergic | Neostigmine | Prostigmin |
| Vasodilator | Epoprostenol | Flolan |

The present invention provides for a range of lipid-based solubilization systems, and particularly liquid crystalline mixtures, and more particularly reversed hexagonal and reversed cubic phase mixtures, whose solubilization properties can be tuned over a broad range. The property that is of importance in the solubilization of actives that have low solubilities in both water and simple lipid-water mixtures is recognized in the present invention to be the concentration and type of polar groups preferentially located in the lipid bilayer or at the polar-apolar interface.

Herein, a phannaceutical active is taken to be of low water-solubility if a therapeutic dose of the active requires more than about 100 ml of water to solubilize it. Similarly, in the present invention a pharmaceutical active is taken to be of low lipid-solubility if a therapeutic dose of the active requires more than about 10 ml octanol in order to solubilize it. The choice of octanol is a natural one since it is the standard solvent in the definition of the important octanol-water partition coefficient, K_{ow}. Further, a compound is considered to be of low lipid-solubility if it is less than 5% by weight soluble in soybean oil.

In addition to solubilizing drugs that are otherwise difficult to solubilize, the non-paraffinic hydrophobes and approaches disclosed in herein can also serve another important role, that of providing a solubilizing matrix into which the pharmaceutically active compound partitions preferentially over water or body fluid (e.g., blood, etc.). For example, certain drugs are not poorly water soluble, yet are more effective in certain situations when they are solubilized in a hydrophobic or amphiphilic environment, as opposed to solubilized in water. In particular, solubilization in a more hydrophobic environment can yield sustained release, or targeted release by holding on to the drug until the matrix reaches the correct site or environment, and/or provide a protective milieu for the drug, or more generally provide a local microenvironment with more favorable chemical or physical properties for production, storage, or application.

As an example, in an Example reported herein, the local anesthetic bupivicaine is solubilized—in its low-solubility, free base form—in a liquid crystal incorporating an essential oil as solubilizing agent, in spite of the fact that the more frequently used hydrochloride salt is water soluble (similar results should be achieved with other local anesthetics such as procaine, prilocaine, cocaine, and tetracaine). This liquid crystal formulation with the free base form so solubilized provides an evironment into which the bupivicaine partitions strongly, since the value of K_{ow} is approximately 1500. This provides an encapsulation approach in which the drug will remain in the matrix even when the processing of the matrix involves contact with excess water, and furthermore will provide for sustained release of the anesthetic, which in the water-solubilized hydrochloride form has a therapeutic half life of only a few hours.

Hydrophobes that Inhibit Drug Efflux

Certain compounds, many of which are non-paraffinic liquids with high octanol-water partition coefficients which do not qualify as surfactants, and most of which in turn comprise at least one polar group that is not operative as a surfactant head group, have been found by the current inventor to induce reversed bicontinuous cubic phases in phosphatidylcholine-water systems. Furthermore, and quite surprisingly, these compounds have been found by the current inventor to show a remarkably strong correlation with the ability, as tablulated by Benet et al. in U.S. Pat. No. 5,716,928, which is herein incorporated by reference, to inhibit the efflux and hydroxylation of cytochrome 3A4 (Cyp3A4) substrates such as cyclosporin. In particular, the following essential oils have been determined by the current inventor to induce a bicontinuous cubic phase in a mixture of the high-PC lecithin “Epikuron 200” (Lucas-Meyer) and water, at a composition of approximately 39% Epikuron, 27% water, and 34% essential oil, at or a few degrees below room temperature: clove bud, ylang-ylang, santalwood, peppermint, eucalyptus, ginger, carrot seed, bay, myrrh, fir needle, patchouli, spearmint, and thyme. The spearmint oil works better in this respect when a portion of the water is replaced by glycerol. In a very surprising correlation, these are precisely the oils that are known to be the strongest inhibitors of the P-glycoprotein/Cyp3A4 efflux system. In contrast, the following oils induce discrete (i.e., non-bicontinuous) cubic phases at the same approximate composition (though typically at slightly lower water concentration): orange, tangerine, wintergreen, fennel, basil, and lemon; these oils are known to be poor inhibitors of the P-gp/Cyp3A4 system; the major components of these oils are either lacking in a polar group entirely (e.g., D-limonene), or have a weakly polar group such as an ester. And those oils which liquify PC-water mixtures at the above composition, even at temperatures of about 15 C., include: citronella, marjoram, and lemongrass; these are known to be poor inhibitors of the P-gp/Cyp3A4 system; typically these oils have aldehydes as their major components. The essential oil component linalool is borderline between the first group and the third, able to induce either a bicontinuous cubic phase or a liquid phase in PC-water systems depending on small changes in composition, and similarly cinnamon (major component: cinnamaldehyde) can have several effects depending on small changes in composition and on the source of the oil.

Examination of the oils which are the best inhibitors—cloves, ylang-ylang, santalwood, peppermint, eucalyptus, ginger, carrot seed, bay, myrrh, fir needle, patchouli, spearmint, and thyme—reveals that each such oil has, as its major component or components, a compound which is a non-paraffinic liquid with a high octanol-water partition coefficient which does not qualify as a surfactant, and comprises at least one polar group that is not operative as a surfactant head group; and furthermore, in the case when the compound has an aldehyde group as the sole polar group, such a compound will not induce a bicontinuous cubic phase in PC-water systems near the above composition nor will it be an effective inhibitor of P-gp/Cyp3A4.

It is apparent from this work that the effect of an essential oil on biomembranes in the body is strongly correlated with its effect on the phospholipid-water system in the test tube, the corrolary being that oils which induce bicontinuous—viz., nanoporous—cubic phases in the test tube are able to induce nanopores, at least transiently, in biomembrane absorption barriers. Since the essential oils are (almost by definition, if not by method of extraction) of low solubility in water, one can assume that when they reach the biomembrane they are in the form of dispersed droplets, so that the local concentration at the point of droplet-biomembrane contact is effectively high, and local patches of a nanoporous microstructure can form as a result. This in turn can provide several means by which the P-glycoprotein-mediated efflux of a pharmaceutically active compound (which normally enhances many-fold the Cyp3A4-mediated hydroxylation of the compound) can be overcome: 1) the nanopore-facilitated apical to basal transport of the essential oil can inhibit the efflux of the active (e.g., cyclosporin) by competitive inhibition; 2) the nonlamellar biomembrane geometry can have a direct effect on efflux-related proteins; 3) the presence of aqueous pores in the biomembrane can allow leakage of ATP, which is required for the function of P-gp. Such effects can even combine synergistically.

The essential oils which fluidize PC-water mixtures in the test tube phase behavior test (resulting in liquid phases, instead of liquid crystalline), as exemplified by citronella, marjoram, and lemongrass oils, do not strongly inhibit the P-gp/Cyp3A4 system. Thus, nanoporosity is of far greater importance than membrane fluidity, in this regard. The conclusion that nanoporosity is the crucial feature is also supported by the fact that the discrete (non-bicontinuous) cubic phase-forming oils are not strong inhibitors, since the discrete cubic phases have very strong curvature (thus ruling out curvature per se as the key feature), but no true porosity.

The current inventor has published a theoretical analysis of surfactant-oil-water phase behavior [Strom, P. and Anderson, D. M. (1992) Langmuir 8:691-702] showing that a polar group on a hydrophobe can have a dramatic effect on the phase behavior of the surfactant-oil-water phase behavior. Thus, the phase behavior results summarized above, in which essential oils characterized by hydrophobes with polar groups yield fundamentally different phase behavior with phospholipids and water than do essential oils without polar groups, are reasonable and not contradictory to known facts.

For the oils which induce bicontinuous cubic phases in PC-water systems, it must be pointed out that most of these convert to reversed hexagonal phases upon reduction of the water concentration, and contrariwise the reversed hexagonal phase will spontaneously convert to a reversed bicontinuous cubic phase upon hydration with water (as may occur, for example, upon application as a drug delivery system, in the body).

Bicontinuous Cubic Phase-Mediated Nanopore Induction in the Delivery of Pharmaceutical Actives

The inventor has found that this same effect of inducing nanopores in biomembranes is a common effect of bicontinuous cubic phases, and is of utility in improving the absorption of pharmaceutical actives whether or not efflux or metabolic (e.g., hydroxylation) proteins are involved. Thus, there is a dramatically and fundamentally different mechanism by which a drug solubilized in a bicontinuous cubic phase can enter a cell, as compared to the same drug solubilized in, say, a liposome. The latter is known to be taken up primarily by endocytosis or pinocytosis, which can be a slow and/or inefficient process. In contrast, the same drug, when solubilized in a reversed bicontinuous cubic phase, need not rely on endocytosis at all—the induction of local, transient nanopores can instead provide a directly accessible route for entry into the cell. By transient, it is meant that the nanopores form and then close in preferably less than an hour and most preferably less than a minute. Furthermore, this is a function of the nanostructure of the phase (the reversed bicontinuous cubic phase), not on the chemistry of the phaseper se: in other words, independently of whether the reversed bicontinuous cubic phase contains essential oil components or hydrophobes with polar groups, the fact that it is in the reversed bicontinuous cubic phase nanostructure, whatever composition yields this, endows the material with the inherent ability to allow for this nanopore-based cell entry mechanism. However, in any case, the presence of components, such as the bicontinuous cubic phase-inducing essential oils listed above, in the vehicle will be most effective and reliable in inducing nanopores in the cell membrane barrier.

Examples 9 and 10 below demonstrate this convincingly. In the case of Example 9, the delivery site is not intestinal but rather neuronal, and the drug, namely bupivacaine, is not subject to the P-gp/Cyp3A4 mechanism discussed in the previous subsection. Nevertheless, the enhancement of cell uptake due to the incorporation of the drug in a cubic phase containing linalool is very dramatic. The fact that the uptake is enhanced is evidenced by the fact that bupivacaine can only exert its anesthetic effect if it is able to enter the cell, since it is known that the drug acts on the drug receptor only on the intracellular portion of the receptor. In the case of Example 10, where the drug is paclitaxel, widely known to be a substrate of the P-gp/Cyp3A4 system, a single cubic phase can accomplish the inhibition of both proteins as well as the induction of nanopores by virtue of its cubic phase nanostructure and its specific composition.

It is also within the realm of this invention for a reversed cubic or reversed hexagonal phase to be formed in situ, from a composition containing a dissolved pharmaceutical active and suitably designed so as to form the desired reversed liquid crystalline phase at the site of cellular uptake. For example, a composition containing dissolved drug, but with less than full saturation with water, could be designed that would swell in body fluids to a reversed cubic phase. Clearly such a material would be within the spirit, and at the site of delivery within the literal language, of this invention.

This nanopore induction mechanism can be of great utility in the delivery of both water-soluble and difficultly-soluble compounds, due in part to the bicontinuous nature of the local, transient patches of biomembrane that facilitate the transport. Thus, the compositions of this invention can be of use in enhancing the delivery, particularly but not limited to the oral delivery, of peptides and proteins (e.g., insulin, erythropoietin, Interferon gamma-1b, Altepase, rh tPA, Darbepoeth alfa, Interferon beta-1a, Coagulation factor IX, Coagulation factor VIIa, rh TNF-alpha, Interferon beta-1b, rH factor VII, rH factor VIII, rH factor IX, Somatropin, Alemtuzumab, Imiglucerase, HbsAg, r TNFR-IgG fragment, rh EPO, Follitropin alpha, Follitropin beta, Glucagon, Trastuzumab, Insulin lispro, rh insulin, Interferon alfacon-1, rh human insulin, Interferon alfa-2b, Anakinra, Insulin glargine, r GM-CSF, rh insulin lispro, r OspA, r IL-2, Rituximab, Oprelvekin, Filgrastim, fh insulin aspart, Muromomab CD3, Peginterferon, rH BsAg, rh EPO, Aldesleukin, Somatrem, Dornase-alpha, Dnase, rh Follicle Stimulating hormone, Retaplase, r tPA, Ribavirin, USP and Interferon alfa-2b recombinant, r HbsAg, Antihemophilic factor, Moroctocog-alfa, Becaplermin, rh PDGF, Infliximab, Abciximab, Reteplase recombinant, Reteplase, r tPA, Hirudin, Rituximab, Interferon alfa-2a, Basiliximab, Palivizumab, Tenecteplase, r HBs Ag, r HBs Ag, Fomivirsen, Daclizumab, etc.), nucleic acids (DNA, RNA, plasmids, antisense compounds, viral-encapsulated nucleic acids, etc.), and small-molecule drugs. In addition to oral delivery, the invention can be of utility in other routes of administration, including but not limited to buccal, intravenous, intramuscular, subcutaneous, intraperitoneal, sublingual, intrathecal, transdermal, intraocular, intranasal, pulmonary, and by direct instillation (e.g., bladder).

Thus, in summary, the inventor has shown that: 1) certain hydrophobes, and in particular certain essential oils, which have non-aldehyde polar groups tend to induce bicontinuous cubic phases in phosphatidylcholine-water systems at a composition of approximately 39% Epikuron, 27% water, and 34% essential oil, at 10-20° C., this being in contrast with oils that either do not have polar groups or are aldehydes and form discrete cubic phases or liquids, respectively; 2) those oils which form bicontinuous cubic phases in phosphatidylcholine-water systems at a composition of approximately 39% Epikuron, 27% water, and 34% essential oil, at 10-20° C., are highly likely to inhibit the Pgp/Cyp3A4 efflux/hydroxylation system, particularly in the small intestine; 3) without wishing to be bound by theory, it is likely that the latter inhibition is due to the formation of local, transient nanoporous domains in the biomembrane barriers of the intestine or other tissue. While U.S. Pat. No. 5,716,928 tabulated inhibitory concentrations of essential oils and their components, nothing was reported in that disclosure on the relationship between chemical structure and activity, nor between PC-oil-water phase behavior and activity. The current work thus provides a foundation for identifying, characterizing, and applying efflux inhibitors for the improved absorption of pharmaceutical actives; 4) this ability to inhibit efflux systems by inducing local, transient pores in cell membranes is an effect common to reversed bicontinuous cubic phases in general; and 5) the same ability to induce local, transient nanopores in cell membranes is applicable to a wide range of drug absorption problems whether or not efflux or metabolic proteins are involved.

Routes of Administration.

The compositions of the present invention may be administered by any of a variety of means which are well known to those of skill in the art. These means include but are not limited to oral (e.g. via pills, tablets, lozenges, capsules, troches, syrups and suspensions, and the like) and non-oral routes (e.g. parenterally, intravenously, intraocularly, transdermally, via inhalation, and the like). The compositions of the present invention are particularly suited for internal (i.e. non-topical) administration. The present invention is especially useful in applications where a difficultly soluble pharmaceutical active is to be delivered internally (i.e. non-topical), including orally and parenterally, wherein said active is to be miscible with a water continuous medium such as serum, urine, blood, mucus, saliva, extracellular fluid, etc. In particular, an important useful aspect of many of the structured fluids of focus herein is that they lend themselves to formulation as water continuous vehicles, typically of low viscosity. The compounds can be administered in a form where they are associated with, and most preferably incorporated within, a said reversed cubic phase or reversed hexagonal phase material, or a combination thereof, that includes a polar solvent, a surfactant, and a non-paraffinic liquid with a high octanol-water partition coefficient which does not qualify as a surfactant. Preferably, the composition administered to a patient is present as a reversed bicontinuous cubic phase and allows delivery of a compound of interest through a biomembrane absorption barrier, such as could be present in a cell, tissue, or organ. Alternatively, co-administration or sequential administration of reversed bicontinous cubic phase materials together with compounds of interest might also be used, whereby the nanoporulation properties discussed in detail above are utilized to enhance delivery of a compound through the biomembrane absorption barrier.

### EXAMPLES

Each of these Examples demonstrates a novel cubic phase composition containing lipid or surfactant, polar solvent (usually water), and a non-paraffinic hydrophobe that does not qualify as a surfactant; furthermore, each Example reports the solubilization of a difficultly-soluble drug in the cubic phase.

### Example 1

The surfactant Pluronic 123, combined with water and a number of non-paraffinic hydrophobes, were found to form reversed cubic phases at specific compositions. The compositions found included the following reversed cubic phase compositions:

Pluronic 123 (47.8%)/orange oil (26.1%)/water (26.1%);

Pluronic 123 (45.7%)/isocugenol (21.7)/water (32.6%); and

Pluronic 123 (47.8%)/lemon oil (26.1%)/water (26.1%).

Furthermore, as exemplified in this Example, these cubic phases are capable of solubilizing drugs of low solubility. Free base bupivacaine (solubility in water less than 0.1% by wt) was made by dissolving 1.00 g of bupivacaine hydrochloride in 24 mL water. An equimolar amount of 1N NaOH was added to precipitate free base bupivacaine. In a glass test tube, 0.280 g free base bupivacaine, 0.685 g water, and 0.679 g linalool were combined and sonicated to break up bupivacaine particles. Then 0.746 g of the surfactant Pluronic P123 was added. The sample was stirred and heated to dissolve the crystalline drug. The sample was centrifuged for fifteen minutes. The sample had formed a highly viscous, clear phase that was optically isotropic in polarizing microscopy. As mentioned above, linalool is a major component of coriander oil, an excipient listed on the FDA list of approved inactive ingredients, and is also the subject of extensive toxicity studies demonstrating its low toxicity.

A second sample was also prepared using the same liquid crystal, then formulating it into microparticles coated with zinc tryptophanate. These bupivacaine-loaded microparticles are suitable for subcutaneous injection, as a slow-release formulation of the local anesthetic with the purpose of prolonging the drug's action and lowering its toxicity profile.

These two samples were then examined by small-angle X-ray scattering. The data were collected on the University of Minnesota 2D small angle x-ray line with copper radiation, Frank mirrors, an evacuated flight path and sample chamber, a Bruker multi-wire area detector, and a sample-to-detector distance of 58 cm (d-spacing range of 172 to 15 angstroms). Since the highest d-spacing observed on this sample was close to the limit of detection with this camera, it was also run on the University of Minnesota 6meter 2D small angle x-ray line with copper radiation, Osmic multi-layer optics, pinhole collimation, an evacuated flight path, helium-filled sample chamber and a Bruker multi-wire area detector and a sample-to-detector distance of 328 cm. At 328 cm the detector has a range of 90 to 700 Angstroms. The first material was loaded into a 1.5 mm i.d. x-ray capillary from Charles Supper Corp. The sample was run at 18 C. The two-dimensional images from the 58 cm distance were integrated with a step size of 0.02 degrees two-theta. Data from the 6-meter line were integrated with a step size of 0.002 degrees two-theta and those plots were overlaid with the runs at the shorter distance, and excellent agreement was obtained between the peak positions recorded with the two cameras.

The x-ray peak analysis software program JADE, by Materials Data Analysis, Inc., was used to analyze the resulting data for the presence and position of peaks. Within that program, the “centroid fit” option was applied.

The SAXS data show Bragg peaks determined by JADE at positions 154.6, 80.6, 61.6, and 46.3 Angstroms. These peaks index to a cubic phase structure of the commonly-observed cubic phase space group of Pn3m (see Pelle Ström and D. M. Anderson, Langmuir, 1992, vol. 8, p. 691 for a detailed discussion of the most commonly observed cubic phase structures and their SAXs patterns). These four peaks in fact index as the (110), (211), (222) and (420) peaks of this space group (#229), with a lattice parameter of 210 Angstroms. The second sample exhibited one peak, at 104.6 Angstroms, which appears to index as the (200) peak of the same lattice. The second sample also showed three peaks with d-spacings less than 25 Angstroms which were clearly due to the crystalline zinc tryptophanate shell.

It is important to point out that only very low levels of bupivacaine can be solubilized in P123-water mixtures without an oil, such as the linalool used here. The hydrochloride form of bupivacaine cannot be dissolved at 2%, and the free base form solubilility is also much lower than the 14% (approx.) level of bupivacaine achieved in this Example.

Isoeugenol is a major component of ylang-ylang oil and other essential oils, and has been the focus of a great deal of toxicity studies demonstrating its low toxicity. Linalool is a major component of coriander oil as well as other essential oils such as cinnamon, and orange oils, and is considered non-paraffinic according to the definition given above because the maximum length of saturated hydrocarbon chain is only 5; the non-paraffinic nature of this compound is underscored by the presence of not only unsaturated bonds but also branching, tertiary carbons, and a hydroxyl group. Linalool has also been the subject of intensive toxicity studies that nearly universally show low toxicity and mutagenicity.

The Pluronics (also called Poloxamers) are a rich class of surfactants that include variants covering a wide range of molecular weights and HLBs. Those with low HLBs are of low water solubility, especially if they are of high MW, and P123 is an example of such a surfactant which nonetheless has a large enough PEG group to form self-association structures under a wide range of conditions. Furthermore its relatively high MW also encourages the formation of liquid crystalline (as opposed to liquid) phases, which is very favorable in the present context. Pluronics are also known to interact strongly with biomembranes so as to enhance cellular absorption of drugs, and may in fact inhibit certain efflux proteins, such as P-glycoprotein and other MDR proteins that are responsible for multidrug resistance. Phosphatidylcholine, for example, has not been shown, or to this author's knowledge even speculated, as performing the latter function in drug-delivery. Pluronics as a class are the subject of a Drug Master File with the FDA, and a number are listed explicitly on the 1996 Inactive Ingredient list as being approved for injectable formulations, indicating their low toxicity.

### Example 2

To begin with, 0.008 g of β-estradiol was combined with 0.203 g of ylang-ylang oil, but did not dissolve, even when heated. After adding 0.497 g of D-alpha tocopheryl polyethylene glycol 1000 succinate (“Vitamin E TPGS”), the estradiol dissolved with gentle heating. Next, 0.322 g of water was added to this solution and the sample was centrifuged for fifteen minutes. A highly viscous, clear phase which was isotropic in polarizing microscopy formed. The same composition, minus the active estradiol, also formed a cubic phase.

For the SAXS analysis, since this material was too viscous to load into a capillary, it was run using a “sandwich” holder; in particular, it was placed inside of a small o-ring sandwiched between thin pieces of Kapton®, a polyimide film.

Bragg peaks were recorded at d-spacings of 123.6, 100.6, 68.8, 49.9, 45.6, and 33.4 Angstroms. These index with good accuracy to a cubic phase Pn3m lattice with a lattice parameter of 174 Angstroms, including the (110), (111), (211), and (222) peaks.

While D-alpha tocopheryl polyethylene glycol 1000 succinate is itself water-soluble, variants of this molecule with shorter PEG chains are of much lower solubility. These surfactants are of great interest in drug-delivery because of their low toxicity, and the fact that they can hydrolyze in the body to yield polyethylene glycol and vitamin E, a powerful antioxidant.

### Example 3

An amount 0.557 g glycerol, 0.314 g sorbitan monooleate, and 0.137 g of essential oil of ginger were combined. After centrifuging for fifteen minutes, this formed a highly viscous, isotropic, slightly yellow, reversed cubic phase on the bottom with a small top layer of excess surfactant and oil. An amount 0.014 g of coenzyme Q10 was dissolved in the cubic phase, yielding a cubic phase with a much deeper yellow-orange color.

This surfactant clearly has advantages over, for example, monoglycerides, which take up very low percentages of oils such as ginger oil, and are thus of little value in solubilizing difficult actives such as Coenzyme Q10. Certain sorbitan esters, such as sorbitan monopalmitate, appear on the 1996 FDA list of Inactive Ingredients as approved for use in injectable products, indicating that they are of very low toxicity.

### Example 4

First, the calcium salt of docusate (2-ethyl hexyl sulfosuccinate) was made by dissolving 10.0 g of the sodium salt of dioctyl sulfosuccinate in 300 mL of water with heating and stirring. Then, 1.27 g of CaCl₂ dissolved in 10.0 g of water was added and a white precipitate formed—indicating the low water solubility of the calcium salt of docusate. This precipitate was dried by vacuum. This low-solubility surfactant was found to form a reversed cubic phase at a composition of: calcium docusate (74%)/linalool (9%)/water (17%). Next, 0.009 g of thioctic acid was dissolved in 0.104 g of linalool by heating. Then 0.901 g of the calcium docusate was added along with 0.210 g of water. Some heating was needed to mix the calcium docusate with the other components of the cubic phase. The sample was centrifuged for fifteen minutes forming an extremely viscous, clear phase that was isotropic in polarizing microscopy.

SAXS peaks were recorded at 30.3, 27.8, and 25.1 Angstroms. This is consistent with a cubic phase of the common type Ia3d (space group #230), with lattice parameter 75 Angstroms, where the observed peak at 30.3 Angstroms compares well with the predicted position of the lowest-order reflection (211), namely 30.6 Angstroms; the next order reflection, (220), has a predicted position of 26.5 Angstroms, and this is probably interpreted as two peaks (27.5 and 25.1) by JADE. An Ia3d cubic phase with lattice parameter 75 Angstroms is perfectly reasonable in view of the well-known cubic phase in the sodium-docusate water system, which also has an Ia3d lattice with lattice parameter of about 80 Angstroms.

Docusates have a long history of safe use in pharmaceutics and other fields, and their anionic charge opens up a range of possibilities in their applications, including enhanced adsorption properties, modulation of their solubilities by counterion substitution, etc.

### Example 5

A reversed hexagonal phase was found at a composition of: polyethylene glycol (5) oleyl ether (37%)/polyethylene glycol (2) oleyl ether (28.5%)/ginger oil (9%)/water (25.5%). Next, 0.008 g of menadione was dissolved in 0.096 g of ginger oil. Next 0.410 g of polyethylene glycol (5) oleyl ether, 0.314 g of polyethylene glycol (2) oleyl ether, and 0.275 g of water were added. The sample was centrifuged to create a viscous, transparent, birefringent phase. Under the microscope, the sample appeared to have hexagonal textures, with a small amount of a liquid phase also being present. SAXS peaks were recorded at 57.4, 33.3, and 29.0 Angstroms, indexing very well to a hexagonal lattice (allowed reflections at d-spacings in the ratio 1:sqrt3:2 . . . ) with a lattice parameter of 57.6 Angstroms. At slightly higher ratios of polyethylene glycol (5) oleyl ether to ginger oil, a reversed cubic phase is observed in this system.

While these particular ethoxylated alcohol surfactants are approved for use only in topical drug-delivery, they have a long history of safe use and represent a class of surfactants, PEGylated lipids, that are known to be of low toxicity and are approved for internal use in many cases.

### Example 6

A mixture of 0.037g of menadione in 0.968 g of ginger oil was heated to dissolve. Then 0.306 of this solution was added to 0.598 g of polyoxyethylene (25) hydrogenated castor oil and 0.308 g water. The sample was stirred to mix and centrifuged for fifteen minutes, producing a viscous, transparent phase which was optically isotropic in polarizing microscopy. The same composition, minus the active menadione, was found to form a reversed cubic phase as well.

Ethoxylated castor oil derivatives such as this are strongly suspected to be inhibitors of certain efflux proteins, such as P-glycoprotein, that limit the absorption of drugs in a variety of cells and induce multidrug resistance. They may also have an effect on biomembranes that will, in a non-specific manner, increase the drug absorption.

### Example 7

The surfactant Pluronic 101 is a very low-HLB, low-solubility surfactant that is approved for internal use according to the 1996 FDA list. A reversed cubic phase was found at a composition of: Pluronic 101 (60%)/ginger oil (15%)/(25%). An amount 0.080 g menadione was heated gently with 1.919 grams of ginger oil to dissolve. An amount 0.149 g of this solution was combined with 0.608 g of Pluronic LI01 and 0.250 g of water. After stirring, the sample was centrifuged for fifteen minutes, producing a viscous, clear phase which appeared optically isotropic in polarizing microscopy. SAXS analysis recorded Bragg peaks in the small-angle range that confirmed the long-range liquid crystalline order of a reversed cubic phase.

### Example 8

The antineoplastic drug paclitaxel (obtained from LKT Labs), in the amount of 13 mg, was dissolved in a mixture of 0.1268 gm of santalwood oil (Cedarvale) and 0.2492 gm of strawberry aldehyde (also known as C-16 aldehyde). To this were added 0.3017 gm deionized water and 0.6179 gm of Pluronic L-122, a low water solubility Pluronic surfactant. This formed a stiff, isotropic cubic phase containing the paclitaxel in solubilized form, that is, in true solution.

### Example 9

The cubic phase of Example 1 was formulated as coated microparticles (as per U.S. Pat. No. 6,482,517 which is herein incorporated by reference), and shown in tests on rats that the formulation strongly enhanced the cellular uptake of bupivacaine. An amount 10.930 gm of Pluronic P123 was combined with 2.698 gm of free base bupivacaine, 10.912 gm of linalool, and 5.447 gm of sterile water, and stirred to form a reversed cubic phase. Of this, 24.982 grams of cubic phase was combined in a flask with 62.807 gm of a diethanolamine-N-acetyltryptophan solution; the latter was prepared by mixing 16.064 gm of diethanolamine, 36.841 gm of sterile water, and 22.491 gm of N-acetyltryptophan and sonicating to combine. The cubic phase/diethanolamine-NAT mixture was first shaken, then homogenized, and finally processed in a Microfluidics microfluidizer to a particle size less than 300 nm. While the material was still in the microfluidizer, 47.219 gm of a 25 wt % zinc acetate solution, and 5.377 gm of diethanolamine were added, and the total mixture microfluidized for 20 runs of 1.5 minutes each. Five ml of a hot (60 C.) mixture of water and sorbitan monopalmitin (6%) was then injected during microfluidization, and next 5 ml of a 14% aqueous solution of albumin. After further microfluidizing, the dispersion was divided into 42 centrifuge tubes of 3.5 ml of dispersion each, and approximately 0.14 gm of Norit activated charcoal was added to each tube, and the tube shaken for 15 minutes on a rocker. Each tube was then centrifuged for 5 minutes in a 6000 rpm tabletop centrifuge. The dispersion was then prefiltered, then filtered at 0.8 microns using Millex AA filters, then placed in a sealed vial and shipped to a facility for animal testing.

The formulation was tested on male Spraque-Dawley rats, weighing 220-250 gm. The animals were maintained under standard conditions, with access to food and water ad libitum. They were briefly anesthetized with halothane during the injection. Sciatic nerve blockage was then tested by administering either the standard 0.5% solution of bupivacaine hydrochloride, or the above cubic phase formulation, by a transcutaneous injection into the popliteal space of the hindlimb. Blockage of thermal nociception was determined by placing the rat on the glass surface of a thermal plantar testing apparatus (Model 336, IITC Inc.), with the surface maintained at 30 C. A mobile radiant heat source located under the glass was focused onto the hindpaw of the rat, and the paw-withdrawal latency recorded by digital timer. The baseline latency was found to be 10 seconds. The rats were tested for latency every 30 minutes.

The sensor blocking effect with the standard 0.5% bupivacaine HCl, at a dose of 3 mg/kg, was found to be 4-5 hours. In contrast, at the same 3 mg/kg dose of the cubic phase formulation, the sensor blocking effect lasted 26 hours. In addition, the latency time itself was greatly increased in the cubic phase case relative to the solution case, indicating a profound pain blockage.

It is known that bupivacaine exerts its action on the cell receptor only when it enters the cell and contacts the intracellular domain of the receptor. Therefore, this experiment demonstrated a strong enhancement of cellular uptake in the presence of the P123-linalool-water cubic phase. Without wishing to be bound by theory, it is believed the the linalool in the cubic phase, as well as the cubic phase itself by virtue of its phase structure, played an active role in enhancing absorption of the drug by inducing nanopores in the biomembrane barriers to absorption.

### Example 10

In this example, the anticancer drug paclitaxel was solubilized in a Pluronic-essential oil-water cubic phase, which was encapsulated by a zinc-NAT shell as in Example 9. The cubic phase was prepared by mixing 0.070 gm of gum benzoin, 0.805 gm of essential oil of sweet basil, and 0.851 gm of oil of ylang-ylang, heating to dissolve the gum benzoin, then adding 265 mg of paclitaxel, 3.257 gm of oil of spearmint, 0.640 gm of strawberry aldehyde, 0.220 gm of ethylhexanoic acid, 1.988 gm of deionized water, and finally 3.909 gm of Pluronic 103. The encapsulating with zinc-NAT was done similarly as in the previous Example, except that short homogenizing was used instead of microfluidizing. The monopalmitin and Norit steps were skipped. The dispersion was placed in vials and sent for testing oral absorption in dogs.

Beagle dogs, 10-12 kg in weight, were cannulated to allow delivery of the formulation directly into the duodenum. Paclitaxel is known to exhibit very low absorption given orally or intraduodenally. Indeed, even in the Taxol® formulation, which includes a large volume of surfactant (Cremophor EL) and ethanol, both of which are membrane fluidizers, the bioavailability is less than about 10%.

Blood levels of paclitaxel were measured at predose, 20 minutes, 40 minutes, 1 hour, 2 hours, 3 hours, 4 hours, 8 hours, 10 hours, and 24 hours. The results for one experiment with the cubic phase formulation were as follows:

| Time point | Blood concentration (ng/ml) |
|---|---|
| 20 min | 79.4 |
| 40 min | 149 |
| 1 hour | 122 |
| 2 hour | 100 |
| 3 hour | 79.5 |
| 4 hour | 70.1 |
| 8 hour | 43.2 |
| 10 hour | 31.1 |
| 24 hour | 17.6 |

These blood levels indicate a high degree of absorption of paclitaxel, and thus a very strong enhancement of absorption due to the cubic phase vehicle in which the paclitaxel was dissolved. Without wishing to be bound by theory, it is believed that the presence of ylang-ylang and spearmint oils, as well as the reversed cubic phase structure itself, effectively induced nanopores in the biomembranes of the intestinal epithelial cells and enhanced the passage of the drug into the cells.

I claim:

1. A composition comprising:
a reversed cubic phase or reversed hexagonal phase material, or a combination thereof, comprised of a polar solvent, a surfactant, and a non-paraffinic liquid with a high octanol-water partition coefficient which does not qualify as a surfactant; and
a compound that is difficultly soluble in water solubilized in said reversed cubic phase or reversed hexagonal phase material, or a combination thereof.

2. A composition as in claim 1 wherein the reversed cubic phase or reversed hexagonal phase material is composed of pharmaceutically acceptable components.

3. A composition as in claim 1 wherein the non-paraffinic liquid comprises a polar group that is not operative as a surfactant head group.

4. A composition as in claim 3 wherein the polar group is selected from the group consisting of a hydroxy, phenolic, aldehyde, ketone, carboxylic acid (in the free acid form), isocyanate, amide, acyl cyanoguanidine, acyl guanylurea, acyl biuret, N,N-dimethylamide, nitrosoalkane, nitroalkane, nitrate ester, nitrite ester, nitrone, nitrosamine, pyridine N-oxide, nitrile, isonitrile, amine borane, amine haloborane, sulfone, phosphine sulfide, arsine sulfide, sulfonamide, sulfonamide methylimine, alcohol (monofunctional), ester (monofunctional), secondary amine, tertiary amine, mercaptan, thioether, primary phosphine, secondary phosphine, and tertiary phosphine.

5. A composition as in claim 1 wherein the non-paraffinic liquid is an essential oil or component thereof.

6. A composition as in claim 3 wherein the non-paraffinic liquid is an essential oil or component thereof.

7. A composition as in claim 1 wherein said compound is relatively more soluble in said reversed cubic phase or reversed hexagonal phase material in the presence of said non-paraffinic liquid than in the absence of said non-paraffinic liquid.

8. A composition as in claim 1 wherein said compound is difficultly-soluble in oil.

9. A composition as in claim 1 wherein the surfactant is of low solubility in water.

10. A composition as in claim 1 wherein said compound is a pharmaceutical active.

11. A composition as in claim 10 wherein said pharmaceutical active is selected from the group consisting of Nandrolone decanoate, Fentanyl citrate, Testosterone, Albendazole, Doxorubicin, Epirubicin, Idarubicin, Valrubicin, Oxybutinin, Amphotericin B, Enalaprilat, Docetaxel, Paclitaxel, Vinblastine, Vincristine, Vinorelbine, Batimastat, Eptifibatide, Tirofiban, Droperidol, Acyclovir, Pentafuside, Saquinavir, Cromolyn, Doxapram, SN-38 (Irinotecan), Topotecan, Hemin, Daunorubicin, Teniposide, Trimetrexate, Octreotride, Leuprolide, Clyclosporin A, Milrinone lactate, Buprenorphine, Nalbuphine, Carboplatin, Cisplatin, Mitoxantrone, Estradiol, Hydroxyprogesterone, L-Thyroxine, Etanercept, Neostigmine, Epoprostenol, Enalapril, Albuterol, Sulfinalol, Nandrolone, Morphine, Aspirin, Testosterone, Hexobarbitol, Cyclexedrine, Niclosamide, Mebendazole, Amphotalide, Retinoic acid, Emetine, Nifedipine, Quinidine, Chloramphenicol, Rifamide, Ampicillin, Erythromycin A, Tetracycline, Ciprofloxacin, Sulfamoxole, Dapsone, Atropine, Warfarin, Nitrazapem, Zometapine, Glyburide, Uzarin, Aspirin, Taxol, Etiposide, Bupivicaine or local anesthetic, and Dantrolene.

12. A composition as in claim 5 wherein the non-paraffinic liquid is selected from the group consisting of benzyl benzoate, peppermint oil, orange oil, spearmint oil, essential oil of ginger, thymol, vanillin, anethole, cinnamon oil, cinnamaldehyde, clove oil, coriander oil, ylang-ylang oil, benzaldehyde, zingerone, carvone, linalool, and menthol.

13. A composition as in claim 1 wherein the polar solvent is selected from the group consisting of water, glycerol, ethylene glycol or propylene glycol, ethylammonium nitrate, acetamide, N-methyl acetamide, dimethylacetamide, and low-molecular weight polyethylene glycol (PEG).

14. A composition as in claim 1 wherein the non-paraffinic liquid has a molecular weight of about 500 or less.

15. A composition as in claim 1 wherein the non-paraffinic liquid has a molecular weight of about 250 or less.

16. A composition as in claim 1 wherein the poorly-water-soluble compound has at least 3 polar groups.

17. A composition as in claim 1 wherein the reversed hexagonal or reversed cubic phase is a component of a pill, tablet, lozenge, capsule, troche, syrup or suspension drug formulation.

18. A composition as in claim 1 wherein the surfactant is chosen from the group consisting of Pluronics, D-alpha tocopheryl polyethylene glycol succinates, sorbitan fatty acid esters, docusate salts, polyethylene glycol oleyl ethers, polyoxyethylene castor oil derivatives, and polyoxyethylene hydrogenated castor oil derivatives.

19. A composition as in claim 1 wherein the reversed hexagonal or reversed cubic phase is tunable.

20. A composition, comprising:
a polar solvent;
a surfactant; and
a non-paraffinic liquid with a polar group that is not operative as a surfactant head group and with a high octanol-water partition coefficient which does not qualify as a surfactant,
wherein the composition is present as a reversed cubic or reversed hexagonal liquid crystalline phase, or a combination thereof.

21. The composition of claim 20 wherein said composition is formulated in internally administrable form and includes only pharmaceutically acceptable components.

22. The composition of claim 20 wherein said composition is present as a reversed bicontinuous cubic phase.

23. The composition of claim 22 wherein said composition is formulated in internally administrable form and includes only pharmaceutically acceptable components.

24. A composition as in claim 20 wherein the polar group is selected from the group consisting of a hydroxy, phenolic, aldehyde, ketone, carboxylic acid (in the free acid form), isocyanate, amide, acyl cyanoguanidine, acyl guanylurea, acyl biuret, N,N-dimethylamide, nitrosoalkane, nitroalkane, nitrate ester, nitrite ester, nitrone, nitrosamine, pyridine N-oxide, nitrile, isonitrile, amine borane, amine haloborane, sulfone, phosphine sulfide, arsine sulfide, sulfonamide, sulfonamide methylimine, alcohol (monofunctional), ester (monofunctional), secondary amine, tertiary amine, mercaptan, thioether, primary phosphine, secondary phosphine, and tertiary phosphine.

25. A composition as in claim 20 wherein the non-paraffinic liquid is an essential oil or component thereof.

26. A composition as in claim 23 wherein the non-paraffinic liquid is an essential oil or component thereof.

27. A composition as in claim 20 wherein the surfactant is of low solubility in water.

28. A composition as in claim 25 wherein the non-paraffinic liquid is selected from the group consisting of benzyl benzoate, peppermint oil, orange oil, spearmint oil, essential oil of ginger, thymol, vanillin, anethole, cinnamon oil, cinnamaldehyde, clove oil, coriander oil, ylang-ylang oil, benzaldehyde, zingerone, carvone, linalool, and menthol.

29. A composition as in claim 20 wherein the polar solvent is selected from the group consisting of water, glycerol, ethylene glycol or propylene glycol, ethylammonium nitrate, acetamide, N-methyl acetamide, dimethylacetamide, and low-molecular weight polyethylene glycol (PEG).

30. A composition as in claim 20 wherein the non-paraffinic liquid has a molecular weight of about 500 or less.

31. A composition as in claim 20 wherein the non-paraffinic liquid has a molecular weight of about 250 or less.

32. A composition as in claim 20 wherein the reversed hexagonal or reversed cubic phase is a component of a pill, tablet, lozenge, capsule, troche, syrup or suspension drug formulation.

33. A composition as in claim 20 wherein the surfactant is chosen from the group consisting of Pluronics, D-alpha tocopheryl polyethylene glycol succinates, sorbitan fatty acid esters, docusate salts, polyethylene glycol oleyl ethers, polyoxyethylene castor oil derivatives, and polyoxyethylene hydrogenated castor oil derivatives.

34. A composition as in claim 20 wherein the reversed hexagonal or reversed cubic phase is tunable.

35. A composition, comprising:
a reversed cubic phase or reversed hexagonal phase material composed of pharmaceutically acceptable components, or a combination thereof, comprised of a polar solvent, a surfactant, and a non-paraffinic liquid having a polar group that is not operative as a surfactant head group, and with a high octanol-water partition coefficient which does not qualify as a surfactant; and
a compound that is difficultly soluble in water solubilized in said reversed cubic phase or reversed hexagonal phase material, or a combination thereof.

36. The composition of claim 35 wherein said composition is present as a reversed bicontinuous cubic phase.

37. The composition of claim 35 wherein said compound is difficultly soluble in oil.

38. The composition of claim 35 wherein said compound is a pharmaceutically active.

39. The composition of claim 1 wherein said composition is present as a reversed bicontinuous cubic phase.

40. A method for solubilizing a difficultly soluble compound comprising the step of incorporating said difficultly soluble compound into a matrix comprised of a reversed cubic or reversed hexagonal liquid crystalline phase material, or a combination thereof, wherein the reversed cubic or reversed hexagonal liquid crystalline phase material comprises a polar solvent, a surfactant, and a non-paraffinic liquid with a high octanol-water partition coefficient which does not qualify as a surfactant.

41. A method for administering a pharmaceutical active compound to a patient, comprising the steps of:
providing said patient with said pharmaceutical active compound associated with a reversed cubic phase or reversed hexagonal phase material, or a combination thereof, and
inducing nanopores in biomembrane absorption barriers in cells or tissues or organs of said patient using said reversed cubic phase or reversed hexagonal phase material, or a combination thereof, wherein said nanopores permit said pharmaceutical active compound to pass therethrough.

42. The method of claim 41 wherein said reversed cubic phase or reversed hexagonal phase material, or a combination thereof is present as a reversed bicontinuous cubic phase.

43. The method of claim 41 wherein nanopores formed in said inducing step are transient.

44. The method of claim 41 wherein said pharmaceutical active compound is difficultly soluble in water.

45. The method of claim 41 wherein said pharmaceutical active compound is difficulty soluble in oil.

46. The method of claim 41 wherein said reversed cubic phase or reversed hexagonal phase material, or a combination thereof, is comprised of a polar solvent, a surfactant, and a non-paraffinic liquid with a high octanol-water partition coefficient which does not qualify as a surfactant.

47. The method of claim 41 wherein said pharmaceutical active compound is selected from the group consisting of Nandrolone decanoate, Fentanyl citrate, Testosterone, Albendazole, Doxorubicin, Epirubicin, Idarubicin, Valrubicin, Oxybutinin, Amphotericin B, Enalaprilat, Docetaxel, Paclitaxel, Vinblastine, Vincristine, Vinorelbine, Batimastat, Eptifibatide, Tirofiban, Droperidol, Acyclovir, Pentafuside, Saquinavir, Cromolyn, Doxapram, SN-38 (Irinotecan), Topotecan, Hemin, Daunorubicin, Teniposide, Trimetrexate, Octreotride, Leuprolide, Clyclosporin A, Milrinone lactate, Buprenorphine, Nalbuphine, Carboplatin, Cisplatin, Mitoxantrone, Estradiol, Hydroxyprogesterone, L-Thyroxine, Etanercept, Neostigmine, Epoprostenol, Enalapril, Albuterol, Sulfinalol, Nandrolone, Morphine, Aspirin, Testosterone, Hexobarbitol, Cyclexedrine, Niclosamide, Mebendazole, Amphotalide, Retinoic acid, Emetine, Nifedipine, Quinidine, Chloramphenicol, Rifamide, Arnpicillin, Erythromycin A, Tetracycline, Ciprofloxacin, Sulfamoxole, Dapsone, Atropine, Warfarin, Nitrazapem, Zometapine, Glyburide, Uzarin, Aspirin, Taxol, Etiposide, Bupivicaine or local anesthetic, and Dantrolene.

48. A method for transporting a compound through a biomembrane absorption barrier, comprising the steps of:
inducing nanopores in said biomembrane absorption barrier using a reversed cubic phase or reversed hexagonal phase material, or a combination thereof, which is associated with said compound; and
passing said compound through said nanopores.

49. The method of claim 48 wherein said compound is difficultly soluble in water.

50. The method of claim 48 wherein said compound is difficulty soluble in oil.

51. The method of claim 48 wherein said reversed cubic phase or reversed hexagonal phase material, or a combination thereof, is comprised of a polar solvent, a surfactant, and a non-paraffinic liquid with a high octanol-water partition coefficient which does not qualify as a surfactant.

52. The method of claim 48 wherein said pharmaceutical active compound is selected from the group consisting of Nandrolone decanoate, Fentanyl citrate, Testosterone, Albendazole, Doxorubicin, Epirubicin, Idarubicin, Valrubicin, Oxybutinin, Amphotericin B, Enalaprilat, Docetaxel, Paclitaxel, Vinblastine, Vincristine, Vinorelbine, Batimastat, Eptifibatide, Tirofiban, Droperidol, Acyclovir, Pentafuside, Saquinavir, Cromolyn, Doxapram, SN-38 (Irinotecan), Topotecan, Hemin, Daunorubicin, Teniposide, Trimetrexate, Octreotride, Leuprolide, Clyclosporin A, Milrinone lactate, Buprenorphine, Nalbuphine, Carboplatin, Cisplatin, Mitoxantrone, Estradiol, Hydroxyprogesterone, L-Thyroxine, Etanercept, Neostigmine, Epoprostenol, Enalapril, Albuterol, Sulfinalol, Nandrolone, Morphine, Aspirin, Testosterone, Hexobarbitol, Cyclexedrine, Niclosamide, Mebendazole, Amphotalide, Retinoic acid, Emetine, Nifedipine, Quinidine, Chloramphenicol, Rifamide, Ampicillin, Erythromycin A, Tetracycline, Ciprofloxacin, Sulfamoxole, Dapsone, Atropine, Warfarin, Nitrazapem, Zometapine, Glyburide, Uzarin, Aspirin, Taxol, Etiposide, Bupivicaine or local anesthetic, and Dantrolene.

53. The method of claim 48 wherein said reversed cubic phase or reversed hexagonal phase material, or a combination thereof is present as a reversed bicontinuous cubic phase.

54. The method of claim 48 wherein nanopores formed in said inducing step are transient.

55. A method for administering a pharmaceutical active compound to a patient, comprising the steps of:
providing said patient with said pharmaceutical active compound;
providing said patient with a reversed cubic phase or reversed hexagonal phase material, or a combination thereof; and
inducing nanopores in biomembrane absorption barriers in cells or tissues or organs of said patient using said reversed cubic phase or reversed hexagonal phase material, or a combination thereof, wherein said nanopores permit said pharmaceutical active compound to pass therethrough.

56. The method of claim 55 wherein said two providing steps are performed together.

57. The method of claim 56 wherein said compound and said reversed cubic phase or reversed hexagonal phase material, or a combination thereof, are associated with each other.

58. The method of claim 55 wherein said two providing steps are performed sequentially.

59. The method of claim 55 wherein said reversed cubic phase or reversed hexagonal phase material, or a combination thereof is present as a reversed bicontinuous cubic phase.

60. The method of claim 55 wherein nanopores formed in said inducing step are transient.

61. The method of claim 55 wherein said pharmaceutical active compound is difficultly soluble in water.

62. The method of claim 55 wherein said pharmaceutical active compound is difficulty soluble in oil.

63. The method of claim 55 wherein said reversed cubic phase or reversed hexagonal phase material, or a combination thereof, is comprised of a polar solvent, a surfactant, and a non-paraffinic liquid with a high octanol-water partition coefficient which does not qualify as a surfactant.

64. The method of claim 55 wherein said pharmaceutical active compound is selected from the group consisting of Nandrolone decanoate, Fentanyl citrate, Testosterone, Albendazole, Doxorubicin, Epirubicin, Idarubicin, Valrubicin, Oxybutinin, Amphotericin B, Enalaprilat, Docetaxel, Paclitaxel, Vinblastine, Vincristine, Vinorelbine, Batimastat, Eptifibatide, Tirofiban, Droperidol, Acyclovir, Pentafuside, Saquinavir, Cromolyn, Doxapram, SN-38 (Irinotecan), Topotecan, Hemin, Daunorubicin, Teniposide, Trimetrexate, Octreotride, Leuprolide, Clyclosporin A, Milrinone lactate, Buprenorphine, Nalbuphine, Carboplatin, Cisplatin, Mitoxantrone, Estradiol, Hydroxyprogesterone, L-Thyroxine, Etanercept, Neostigmine, Epoprostenol, Enalapril, Albuterol, Sulfinalol, Nandrolone, Morphine, Aspirin, Testosterone, Hexobarbitol, Cyclexedrine, Niclosamide, Mebendazole, Amphotalide, Retinoic acid, Emetine, Nifedipine, Quinidine, Chloramphenicol, Rifamide, Ampicillin, Erythromycin A, Tetracycline, Ciprofloxacin, Sulfamoxole, Dapsone, Atropine, Warfarin, Nitrazapem, Zometapine, Glyburide, Uzarin, Aspirin, Taxol, Etiposide, Bupivicaine or local anesthetic, and Dantrolene.

65. A method for transporting a compound through a biomembrane absorption barrier, comprising the steps of:
inducing nanopores in said biomembrane absorption barrier using a reversed cubic phase or reversed hexagonal phase material, or a combination thereof; and
passing said compound through said nanopores.

66. The method of claim 65 wherein said compound and said reversed cubic phase or reversed hexagonal phase material, or a combination thereof, are associated with each other.

67. The method of claim 65 wherein said compound and said reversed cubic phase or reversed hexagonal phase material, or a combination thereof, are separate from each other.

68. The method of claim 65 wherein said compound is difficultly soluble in water.

69. The method of claim 65 wherein said compound is difficulty soluble in oil.

70. The method of claim 65 wherein said reversed cubic phase or reversed hexagonal phase material, or a combination thereof, is comprised of a polar solvent, a surfactant, and a non-paraffinic liquid with a high octanol-water partition coefficient which does not qualify as a surfactant.

71. The method of claim 65 wherein said pharmaceutical active compound is selected from the group consisting of Nandrolone decanoate, Fentanyl citrate, Testosterone, Albendazole, Doxorubicin, Epirubicin, Idarubicin, Valrubicin, Oxybutinin, Amphotericin B, Enalaprilat, Docetaxel, Paclitaxel, Vinblastine, Vincristine, Vinorelbine, Batimastat, Eptifibatide, Tirofiban, Droperidol, Acyclovir, Pentafuside, Saquinavir, Cromolyn, Doxapram, SN-38 (Irinotecan), Topotecan, Hemin, Daunorubicin, Teniposide, Trimetrexate, Octreotride, Leuprolide, Clyclosporin A, Milrinone lactate, Buprenorphine, Nalbuphine, Carboplatin, Cisplatin, Mitoxantrone, Estradiol, Hydroxyprogesterone, L-Thyroxine, Etanercept, Neostigmine, Epoprostenol, Enalapril, Albuterol, Sulfinalol, Nandrolone, Morphine, Aspirin, Testosterone, Hexobarbitol, Cyclexedrine, Niclosamide, Mebendazole, Amphotalide, Retinoic acid, Emetine, Nifedipine, Quinidine, Chloramphenicol, Rifamide, Ampicillin, Erythromycin A, Tetracycline, Ciprofloxacin, Sulfamoxole, Dapsone, Atropine, Warfarin, Nitrazapem, Zometapine, Glyburide, Uzarin, Aspirin, Taxol, Etiposide, Bupivicaine or a local anesthetic, and Dantrolene.

72. The method of claim 65 wherein said reversed cubic phase or reversed hexagonal phase material, or a combination thereof is present as a reversed bicontinuous cubic phase.

73. The method of claim 65 wherein nanopores formed in said inducing step are transient.

74. The composition of claim 3 wherein said non-paraffinic liquid is an essential oil or a component thereof selected from the group consisting of clove bud, ylang-ylang, santalwood, peppermint, eucalyptus, ginger, carrot seed, bay, myrrh, fir needle, patchouli, spearmint, and thyrne.

75. The composition of claim 35 wherein said non-paraffinic liquid is an essential oil or a component thereof selected from the group consisting of clove bud, ylang-ylang, santalwood, peppermint, eucalyptus, ginger, carrot seed, bay, myrrh, fir needle, patchouli, spearmint, and thyme.

76. The method of claim 46 wherein said non-paraffinic liquid is an essential oil or a component thereof selected from the group consisting of clove bud, ylang-ylang, santalwood, peppermint, eucalyptus, ginger, carrot seed, bay, myrrh, fir needle, patchouli, spearmint, and thyme.

77. The method of claim 51 wherein said non-paraffinic liquid is an essential oil or a component thereof selected from the group consisting of clove bud, ylang-ylang, santalwood, peppermint, eucalyptus, ginger, carrot seed, bay, myrrh, fir needle, patchouli, spearmint, and thyme.

78. The method of claim 63 wherein said non-paraffinic liquid is an essential oil or a component thereof selected from the group consisting of clove bud, ylang-ylang, santalwood, peppermint, eucalyptus, ginger, carrot seed, bay, myrrh, fir needle, patchouli, spearmint, and thyme.

79. The method of claim 70 wherein said non-paraffinic liquid is an essential oil or a component thereof selected from the group consisting of clove bud, ylang-ylang, santalwood, peppermint, eucalyptus, ginger, carrot seed, bay, myrrh, fir needle, patchouli, spearmint, and thyme.
